(19) United States
(12) Patent Application Publication
(10) Pub. No.: US 2026/0133192 A1
(43) Pub. Date: May 14, 2026

(54) SMALL CELL LUNG CANCER TUMOR ANTIGENS AND USES THEREOF

(71) Applicant: Fred Hutchinson Cancer Center, Seattle, WA (US)

(72) Inventors: Kristin LASTWIKA, Seattle, WA (US); Paul LAMPE, Seattle, WA (US); Ashley MCGARRY HOUGHTON, Seattle, WA (US)

(21) Appl. No.: 19/119,985

(22) PCT Filed: Oct. 10, 2023
(86) PCT No.: PCT/US2023/034841

§ 371 Date: Apr. 10, 2025

Related U.S. Application Data
(60) Provisional application No. 63/415,142, filed on Oct. 11, 2022.

(51) Int. Cl. G01N 33/564; G01N 33/575; G01N 33/5752

(52) U.S. Cl. CPC G01N 33/564; G01N 33/5752; G01N 33/57585

(57) ABSTRACT

Disclosed herein are high performance biomarkers and panels for small cell lung cancer (SCLC) early detection useful for identifying, diagnosing, and treating SCLC patients at an early stage. The method of diagnosing and treating SCLC in a subject can include detecting presence of autoantibodies that specifically bind one or more epitopes of antigens in a biological sample obtained from the subject, wherein the one or more epitopes of the antigens comprises a post-translational modification; diagnosing the subject with SCLC when the presence of the autoantibodies is detected in the biological sample; and treating the SCLC in the subject by administering a chemotherapy regimen, an immunotherapy regimen, an antibody, performing a surgery, and/or radiotherapy. In one example, the post-translational modifications targeted by the autoantibodies include citrullination, isoaspartylation and/or cancer-specific glycosylation.

### CROSS REFERENCE TO RELATED APPLICATIONS

This application claims the benefit under 35 USC § 119(e) of U.S. Provisional Application No. 63/415,143 filed Oct. 11, 2022, which is incorporated herein by reference in its entirety for all purposes.

### ACKNOWLEDGEMENT OF GOVERNMENT SUPPORT

This invention was made with government support under CA268066, CA243328, CA186157, and CA281801 awarded by the National Institutes of Health. The government has certain rights in the invention.

### REFERENCE TO A SEQUENCE LISTING

This application incorporates by reference a computer readable Sequence Listing in ST.26 XML format, titled 23-032-WO-PCT_Sequence, created on Sep. 25, 2023 and containing 98,924 bytes.

### FIELD OF THE INVENTION

This disclosure relates to the field of medicine and in particular, to autoantibodies and autoantibody-autoantigen complexes as biomarkers of small cell lung cancer (SCLC), and methods of use thereof.

### BACKGROUND

Small cell lung cancer (SCLC) is the 6^{th} leading cause of cancer-related deaths in the United States with fewer than 6% of patients surviving 5-years after diagnosis (Siegel, et al., 2022, A Cancer Journal for Clinicians 72, 7-33; Wang, et al., 2017, Sci Rep 7, 1339). SCLC accounts for approximately 10-15% of lung cancers and shares one very important characteristic with essentially all solid tumor malignancies: early detection of the cancer leads to improved survival metrics. However, a critical barrier to effective and life-saving treatment is the lack of early detection, as 70% of SCLC patients are diagnosed with metastatic disease (Alvarado-Luna and Morales-Espinosa, 2016, Transl Lung Cancer Res 5, 26-38). This is likely a result of the aggressive nature and mediastinal location of SCLC, such that annual imaging is not sufficient. There currently is no blood test recommended for lung cancer early detection. Since the majority of SCLC patients are diagnosed at extensive stage where current treatment options offer limited benefits, many investigators have not appreciated the fact that nearly approximately 20% of limited stage SCLC patients can be cured with conventional cytotoxic chemotherapy. Furthermore, surgical resection, generally not considered for SCLC, can be curative when combined with chemotherapy for highly selected and very early stage patients. The low-dose CT screening protocols that have proven effective for non-small cell lung cancer have not displayed benefits in SCLC. As such, assays and methods for identifying SCLC in the early stage are needed.

More than any other cancer type, SCLC has a strong association with a group of autoimmune diseases called paraneoplastic neurological syndromes (PNS) (Höftberger, et al., 2015, Curr Opin Oncol 27, 489-495). Many PNS are considered clinical manifestations of autoantibody (AAb) production against neuroendocrine antigens expressed by tumor cells and cross-reactive in the central nervous system (Anwar, et al., 2019, Ann Transl Med 7), but the mechanistic basis and scope of AAb production in SCLC remains poorly understood. Strikingly, PNS symptoms precede cancer diagnosis in 80% of affected patients and result in increased detection of early-stage SCLC and improved overall survival (Sebastian, et al., 2019, Journal of Thoracic Oncology 14, 1878-1880; Honnorat and Antoine, 2007, Orphanet Journal of Rare Diseases 2, 22). Moreover, while PNS diagnoses are rare, AAbs to PNS antigens can be found in a substantial fraction of SCLC patients that do not display autoimmune symptoms (Kazarian and Laird-Offringa, 2011, Mol Cancer 10, 33). This unique relationship of AAb production during SCLC tumorigenesis suggests AAbs to non-PNS antigens could exist and more broadly serve as biomarkers of SCLC.

SCLC-specific AAbs that do not cause overt autoimmune diseases like PNS are likely to be initiated by tumor alterations that increase the immunogenicity of self-antigens. Unfortunately, sequence or structure-based methods for predicting immunogenic epitopes in silico have limited power (Jespersen, et al., 2019, Frontiers in Immunology 10) and current experimental methods for AAb discovery have several limitations. Many approaches for AAb detection utilize capture reagents that have been fixed or linearized and thus exclude AAbs targeting conformational protein epitopes. Recombinant proteins produced in non-human species like bacteria or yeast have been used as conformational capture antigens but fail to detect AAbs targeting human-specific post-translational modifications (PTMs) or disease-specific neoepitopes (Doyle and Mamula, 2001, Trends in Immunology 22, 443-449). Methods that aim to comprehensively map potential epitopes like serum epitope repertoire analysis (Kamath, et al., 2020, Sci Rep 10, 5294) or tumor proteome fractionation (Qiu, et al., 2008, J Clin Oncol 26, 5060-5066) can be successful in identifying disease-specific AAbs but require labor intensive deciphering of the cognate antigen on the back end. Therefore, alternative high-dimensional approaches to capture disease-relevant AAbs are needed.

Early diagnosis of SCLC can meaningfully impact the current standard of care treatment. Current treatment options include chemotherapy with platinum-etoposide, given concurrently with thoracic irradiation in patients with limited stage disease and chemotherapy alone in those with extensive stage (Corso, et al., 2015, J Clin Oncol 33, 4240-4246). Prophylactic cranial irradiation is recommended for patients who have responded to initial therapy, as it not only decreases the risk of brain metastases but also improves overall survival. Surgical resection of SCLC presenting as a solitary pulmonary nodule during early stage disease has shown promising results (Kreisman, et al., 1992, Chest 101, 225-31).

Adoptive immunotherapy, which involves the transfer of autologous antigen-specific T cells generated ex vivo, is a promising strategy to treat cancer. Novel specificities in T cells have been successfully generated through the genetic transfer of transgenic T cell receptors or chimeric antigen receptors (CARs) (Jena, et al., 2010, Blood 116(7), 1035-44). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and heavy chain variable fragments of a monoclonal antibody joined by a flexible linker. The signaling domains for first generation CARs are derived from the cytoplasmic region of the CD3zeta or the Fc receptor gamma chains. Engineered cells expressing chimeric antigen receptors that target SCLC would be useful in therapeutic settings in which specific targeting and T cell-mediated killing of SCLC cells is desired.

### BRIEF SUMMARY OF THE INVENTION

Disclosed herein are methods of diagnosing and treating small cell lung cancer in a subject. According to embodiments of the present disclosure, a large format antibody array that captures circulating AAb-antigen (AAb-Ag) complexes from human plasma is disclosed. Isolating AAb-Ag complexes directly from patients uniquely enables detection of AAbs bound to their native tumor-derived epitopes ex vivo. In multiple independent cohorts of SCLC, a set of antigens with their corresponding AAb that are upregulated before diagnosis and in both limited and extensive stage diseases is identified and validated. These AAbs identified novel tumor-associated antigens, some of which were expressed on the cell surface in most SCLC tumors examined. The detailed analysis revealed that some of these proteins contained immunogenic PTMs including citrullination, isoaspartylation and cancer-specific glycosylation that when mimicked with synthetic PTM-containing peptides, confirmed binding to SCLC patient AAb.

Also disclosed herein is a unique, high-throughput approach to identify tumor-specific AAbs that could easily be applied to AAb discovery in other diseases, such as autoimmunity or infection. A combination of the validated AAbs yielded remarkable utility in a risk prediction model for SCLC that may further be improved by incorporating smoking pack years. If applied during lung cancer screening, this model has the potential to meaningfully impact the overall survival of this deadly disease through early detection of SCLC and current standard of care treatment.

In one aspect, the present disclosure provides a method of treating small cell lung cancer (SCLC) in a subject, the method comprising: (a) administering a chemotherapy regimen to the subject; (b) administering an immunotherapy regimen to the subject; (c) administering an antibody or antigen-binding fragment thereof to the subject, (d) performing surgical resection of the SCLC in the subject; and/or (e) contacting the subject with radiotherapy targeting the SCLC, wherein the subject has autoantibodies that specifically bind one or more epitopes of one or more antigens selected from the group consisting of CDH5, CD133, SPINK1, CDH23, NLRP7, TFRC, SPINT2, NADSYN1, HIF1A, GRAP2, MAPRE1, INHA, PTEN, CTSB, B3GNT6, PLD3, TIMP2, NUDT2, ANAPC2, GPLD1, PTPRU and CA9, and wherein the one or more epitopes of the antigen comprises a post-translational modification (PTM).

In some embodiments of the method, the subject has autoantibodies that specifically bind two or more epitopes of the one or more antigens. In some cases, the subject has autoantibodies that specifically bind three or more epitopes of the one or more antigens. In some cases, the subject has autoantibodies that specifically bind four or more epitopes of the one or more antigens. In some cases, the subject has autoantibodies that specifically bind five or more epitopes of the one or more antigens.

In some embodiments of the method, the one or more antigens is selected from the group consisting of SPINK1, TFRC, GRAP2, TIMP2 and PLD3. In some cases, the one or more antigens is selected from the group consisting of SPINK1, GRAP2, TIMP2 and PLD3. In some cases, the one or more antigens is selected from the group consisting of SPINT2, SPINK1, TIMP2 and TFRC.

In some embodiments of the method, the one or more epitopes is selected from the group consisting of SEQ ID NOs: 1-21. In some cases, the PTM comprises citrullination, isoaspartylation and/or glycosylation. In some cases, the PTM comprises citrullination and/or isoaspartylation. In some cases, the one or more epitopes is selected from the group consisting of SEQ ID NOs: 22-46.

In one aspect, the present disclosure provides a method of diagnosing and treating small cell lung cancer (SCLC) in a subject, the method comprising: (a) detecting for presence of one or more autoantibodies that specifically bind one or more epitopes of one or more antigens in a biological sample obtained from the subject, wherein the one or more antigens is selected from the group consisting of CDH5, CD133, SPINK1, CDH23, NLRP7, TFRC, SPINT2, NADSYN1, HIF1A, GRAP2, MAPRE1, INHA, PTEN, CTSB, B3GNT6, PLD3, TIMP2, NUDT2, ANAPC2, GPLD1, PTPRU and CA9, and wherein the one or more epitopes of the antigen comprises a post-translational modification (PTM); (b) diagnosing the subject with SCLC when the presence of the one or more autoantibodies is detected in the biological sample obtained from the subject; and (c) treating the SCLC in the subject by: (i) administering a chemotherapy regimen to the subject; (ii) administering an immunotherapy regimen to the subject; (iii) administering an antibody or antigen-binding fragment thereof to the subject; (iv) performing surgical resection of the SCLC in the subject; and/or (v) contacting the subject with radiotherapy targeting the SCLC. In some embodiments, the method further comprises obtaining the biological sample from the subject. In some cases, the biological sample is serum or plasma.

In some embodiments of the method, diagnosing the subject comprises diagnosing the subject with SCLC when the presence of autoantibodies that bind two or more epitopes of the one or more antigens is detected in the biological sample. In some cases, diagnosing the subject comprises diagnosing the subject with SCLC when the presence of autoantibodies that bind three or more epitopes of the one or more antigens is detected in the biological sample. In some cases, diagnosing the subject comprises diagnosing the subject with SCLC when the presence of autoantibodies that bind four or more epitopes of the one or more antigens is detected in the biological sample. In some cases, diagnosing the subject comprises diagnosing the subject with SCLC when the presence of autoantibodies that bind five or more epitopes of the one or more antigens is detected in the biological sample.

In some embodiments of the method discussed immediately above, the one or more antigens is selected from the group consisting of SPINK1, TFRC, GRAP2, TIMP2 and PLD3. In some cases, the one or more antigens is selected from the group consisting of SPINK1, GRAP2, TIMP2 and PLD3. In some cases, the one or more antigens is selected from the group consisting of SPINT2, SPINK1, TIMP2 and TFRC.

In some embodiments of the method discussed immediately above, the one or more epitopes is selected from the group consisting of SEQ ID NOs: 1-21. In some cases, the PTM comprises citrullination, isoaspartylation and/or glycosylation. In some cases, the PTM comprises citrullination and/or isoaspartylation. In some cases, the one or more epitopes is selected from the group consisting of SEQ ID NOs: 22-46. In some cases, the subject is a human.

In some embodiments of the methods discussed herein, the chemotherapy regimen comprises one or more cycles of cisplatin or carboplatin with etoposide.

In some embodiments, the immunotherapy regimen comprises a chimeric antigen receptor T cell that specifically binds one or more of the epitopes. In some cases, the immunotherapy regimen comprises an antibody that binds one or more of the epitopes. In some cases, the antibody is conjugated to a cytotoxic agent. In some embodiments, the immunotherapy regimen comprises a bispecific antibody that binds one or more of the epitopes and a T-cell antigen. In some cases, the T-cell antigen is CD3.

In one aspect, the treatment methods comprises administration of an antibody or antigen binding fragment thereof to the subject. In some embodiments, the antibody or antigen binding fragment thereof specifically binds one or more epitopes of one or more antigens, wherein the one or more antigens is selected from the group consisting of CDH5, CD133, SPINK1, CDH23, NLRP7, TFRC, SPINT2, NADSYN1, HIF1A, GRAP2, MAPRE1, INHA, PTEN, CTSB, B3GNT6, PLD3, TIMP2, NUDT2, ANAPC2, GPLD1, PTPRU and CA9, and wherein the one or more epitopes of the antigen comprises a post-translational modification (PTM).

In some embodiments, the antibody or the antigen binding fragment thereof comprises three heavy chain complementarity determining regions (CDRs) contained within a heavy chain variable region (HCVR), and three light chain CDRs contained within a light chain variable region (LCVR), wherein the amino acid sequences of the HCVR/LCVR, respectively, comprise the amino acid sequences selected from the group consisting of SEQ ID NOs: 47/51, 55/59, 63/67, 71/75, and 79/83.

In some embodiments, the antibody or antigen-binding fragment comprises HCDR1-HCDR2-HCDR3-LCDR1-LCDR2-LCDR3 domains comprising the amino acid sequences, respectively, selected from the group consisting of SEQ ID NOs: 48-49-50-52-53-54, 56-57-58-60-61-62, 64-65-66-68-69-70, 72-73-74-76-77-78, and 80-81-82-84-85-86.

In some embodiments, the antibody or antigen-binding fragment thereof comprises a heavy chain variable region (HCVR) and a light chain variable region (LCVR), wherein the amino acid sequences of the HCVR/LCVR, respectively, are selected from the group consisting of SEQ ID NOs: 47/51, 55/59, 63/67, 71/75, and 79/83.

In some embodiments, the antibody or antigen binding fragment thereof binds to an epitope of TFRC-4 comprising a PTM, and wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 79, and three light chain CDRs (LCDR1, LCDR2, and LCDR3) contained within a light chain variable region (LCVR) comprising the amino acid sequence of SEQ ID NO: 83.

In some embodiments, the antibody or antigen binding fragment comprises HCDR1-HCDR2-HCDR3-LCDR1-LCDR2-LCDR3 domains comprising the amino sequences of SEQ ID NOs: 80-81-82-84-85-86, respectively.

In some embodiments, the antibody or antigen-binding fragment thereof comprises a HCVR comprising the amino acid sequence of SEQ ID NO: 79, and a LCVR comprising the amino acid sequence of SEQ ID NO: 83. In some embodiments, the epitope comprises the amino acid sequence of SEQ ID NO: 39.

In some embodiments, the antibody or antigen binding fragment thereof binds to an epitope of CA9-3 comprising a PTM, and wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 71, and three light chain CDRs (LCDR1, LCDR2, and LCDR3) contained within a light chain variable region (LCVR) comprising the amino acid sequence of SEQ ID NO: 75.

In some embodiments, the antibody or antigen binding fragment comprises HCDR1-HCDR2-HCDR3-LCDR1-LCDR2-LCDR3 domains comprising the amino sequences of SEQ ID NOs: 72-73-74-76-77-78, respectively. In some embodiments, the antibody or antigen-binding fragment thereof comprises a HCVR comprising the amino acid sequence of SEQ ID NO: 71, and a LCVR comprising the amino acid sequence of SEQ ID NO: 75. In some embodiments, the epitope comprises the amino acid sequence of SEQ ID NO: 45.

In some embodiments, the antibody or antigen binding fragment thereof binds to an epitope of TFRC-5 comprising a PTM, and wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 47, and three light chain CDRs (LCDR1, LCDR2, and LCDR3) contained within a light chain variable region (LCVR) comprising the amino acid sequence of SEQ ID NO: 51.

In some embodiments, the antibody or antigen binding fragment comprises HCDR1-HCDR2-HCDR3-LCDR1-LCDR2-LCDR3 domains comprising the amino sequences of SEQ ID NOs: 48-49-50-52-53-54, respectively. In some embodiments, the antibody or antigen-binding fragment thereof comprises a HCVR comprising the amino acid sequence of SEQ ID NO: 47, and a LCVR comprising the amino acid sequence of SEQ ID NO: 51. In some embodiments, the epitope comprises the amino acid sequence of SEQ ID NO: 40.

In some embodiments, the antibody or antigen binding fragment thereof binds to an epitope of TFRC-1 or TFRC-2 comprising a PTM, and wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 55, and three light chain CDRs (LCDR1, LCDR2, and LCDR3) contained within a light chain variable region (LCVR) comprising the amino acid sequence of SEQ ID NO: 59.

In some embodiments, the antibody or antigen binding fragment comprises HCDR1-HCDR2-HCDR3-LCDR1-LCDR2-LCDR3 domains comprising the amino sequences of SEQ ID NOs: 56-57-58-60-61-62, respectively. In some embodiments, the antibody or antigen-binding fragment thereof comprises a HCVR comprising the amino acid sequence of SEQ ID NO: 55, and a LCVR comprising the amino acid sequence of SEQ ID NO: 59. In some embodiments, the epitope comprises the amino acid sequence of SEQ ID NO: 36 or SEQ ID NO: 37.

In some embodiments, the antibody or antigen binding fragment thereof binds to an epitope of SPINK1-1 comprising a PTM, and wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 63, and three light chain CDRs (LCDR1, LCDR2, and LCDR3) contained within a light chain variable region (LCVR) comprising the amino acid sequence of SEQ ID NO: 67.

In some embodiments, the antibody or antigen binding fragment comprises HCDR1-HCDR2-HCDR3-LCDR1-LCDR2-LCDR3 domains comprising the amino sequences of SEQ ID NOs: 64-65-66-68-69-70, respectively. In some embodiments, the antibody or antigen-binding fragment thereof comprises a HCVR comprising the amino acid sequence of SEQ ID NO: 63, and a LCVR comprising the aminoi acid sequence of SEQ ID NO: 67. In some embodiments, the epitope comprises the amino acid sequence of SEQ ID NO: 29.

In one aspect, the present disclosure provides a biomarker detection panel for small cell lung cancer (SCLC) comprising one or more peptides comprising an epitope of from 7 to 50 amino acids in length from one or more antigens selected from the group consisting of CDH5, CD133, SPINK1, CDH23, NLRP7, TFRC, SPINT2, NADSYN1, HIF1A, GRAP2, MAPRE1, INHA, PTEN, CTSB, B3GNT6, PLD3, TIMP2, NUDT2, ANAPC2, GPLD1, PTPRU and CA9, wherein the epitope comprises a post-translational modification (PTM) bound by an autoantibody.

In some embodiments, the biomarker detection panel is capable of detecting SCLC in a subject with a sensitivity and specificity of at least 60%. In some cases, the biomarker detection panel is capable of detecting SCLC in a subject with a sensitivity and specificity of at least 70% or 75%. In some cases, the biomarker detection panel is capable of detecting SCLC in a subject with a sensitivity and specificity of at least 80% or 85%. In some cases, the biomarker detection panel is capable of detecting SCLC in a subject with a sensitivity and specificity of at least 90% or 95%.

In some embodiments, the biomarker detection panel comprises two or more epitopes from the one or more antigens. In some cases, the biomarker detection panel comprises three or more epitopes of the one or more antigens. In some cases, the biomarker detection panel comprises four or more epitopes of the one or more antigens. In some cases, the biomarker detection panel comprises five or more epitopes of the one or more antigens.

In some embodiments of the biomarker detection panel discussed above, the one or more antigens is selected from the group consisting of SPINK1, TFRC, GRAP2, TIMP2 and PLD3. In some cases, the one or more antigens is selected from the group consisting of SPINK1, GRAP2, TIMP2 and PLD3. In some cases, the one or more antigens is selected from the group consisting of SPINT2, SPINK1, TIMP2 and TFRC.

In some embodiments of the biomarker detection panel discussed above, the one or more epitopes is selected from the group consisting of SEQ ID NOs: 1-21. In some cases, the PTM comprises citrullination, isoaspartylation and/or glycosylation. In some cases, the PTM comprises citrullination and/or isoaspartylation. In some cases, the one or more epitopes is selected from the group consisting of SEQ ID NOs: 22-46.

In some embodiments, the biomarker detection panel is for detecting an autoantibody that binds the one or more epitopes of the one or more antigens in a biological sample obtained from a subject.

In one aspect, this disclosure relates to an isolated antibody or antigen binding fragment thereof that binds to an epitope of TFRC-4 comprising a PTM, wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 79, and three light chain CDRs (LCDR1, LCDR2, and LCDR3) contained within a light chain variable region (LCVR) comprising the amino acid sequence of SEQ ID NO: 83.

In some embodiments, the antibody or antigen binding fragment comprises HCDR1-HCDR2-HCDR3-LCDR1-LCDR2-LCDR3 domains comprising the amino sequences of SEQ ID NOs: 80-81-82-84-85-86, respectively. In some embodiments, the antibody or antigen-binding fragment thereof comprises a HCVR comprising the amino acid sequence of SEQ ID NO: 79, and a LCVR comprising the amino acid sequence of SEQ ID NO: 83. In some embodiments, the epitope comprises the amino acid sequence of SEQ ID NO: 39.

In one aspect, this disclosure relates to an antibody or antigen binding fragment thereof that binds to an epitope of CA9-3 comprising a PTM, wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 71, and three light chain CDRs (LCDR1, LCDR2, and LCDR3) contained within a light chain variable region (LCVR) comprising the amino acid sequence of SEQ ID NO: 75.

In some embodiments, the antibody or antigen binding fragment comprises HCDR1-HCDR2-HCDR3-LCDR1-LCDR2-LCDR3 domains comprising the amino sequences of SEQ ID NOs: 72-73-74-76-77-78, respectively. In some embodiments, the antibody or antigen-binding fragment thereof comprises a HCVR comprising the amino acid sequence of SEQ ID NO: 71, and a LCVR comprising the amino acid sequence of SEQ ID NO: 75. In some embodiments, the epitope comprises the amino acid sequence of SEQ ID NO: 45.

In one aspect, this disclosure relates to an antibody or antigen binding fragment thereof that binds to an epitope of TFRC-5 comprising a PTM, wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 47, and three light chain CDRs (LCDR1, LCDR2, and LCDR3) contained within a light chain variable region (LCVR) comprising the amino acid sequence of SEQ ID NO: 51.

In some embodiments, the antibody or antigen binding fragment comprises HCDR1-HCDR2-HCDR3-LCDR1-LCDR2-LCDR3 domains comprising the amino sequences of SEQ ID NOs: 48-49-50-52-53-54, respectively. In some embodiments, the antibody or antigen-binding fragment thereof comprises a HCVR comprising the amino acid sequence of SEQ ID NO: 47, and a LCVR comprising the amino acid sequence of SEQ ID NO: 51. In some embodiments, the epitope comprises the amino acid sequence of SEQ ID NO: 40.

In one aspect, this disclosure relates to an antibody or antigen binding fragment thereof that binds to an epitope of TFRC-1 or TFRC-2 comprising a PTM, wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 55, and three light chain CDRs (LCDR1, LCDR2, and LCDR3) contained within a light chain variable region (LCVR) comprising the amino acid sequence of SEQ ID NO: 59.

In some embodiments, the antibody or antigen binding fragment comprises HCDR1-HCDR2-HCDR3-LCDR1-LCDR2-LCDR3 domains comprising the amino sequences of SEQ ID NOs: 56-57-58-60-61-62, respectively. In some embodiments, the antibody or antigen-binding fragment thereof comprises a HCVR comprising the amino acid sequence of SEQ ID NO: 55, and a LCVR comprising the amino acid sequence of SEQ ID NO: 59. In some embodiments, the epitope comprises the amino acid sequence of SEQ ID NO: 36 or SEQ ID NO: 37.

In one aspect, this disclosure relates to an antibody or antigen binding fragment thereof that binds to an epitope of SPINK1-1 comprising a PTM, wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 63, and three light chain CDRs (LCDR1, LCDR2, and LCDR3) contained within a light chain variable region (LCVR) comprising the amino acid sequence of SEQ ID NO: 67.

In some embodiments, the antibody or antigen binding fragment comprises HCDR1-HCDR2-HCDR3-LCDR1-LCDR2-LCDR3 domains comprising the amino sequences of SEQ ID NOs: 64-65-66-68-69-70, respectively. In some embodiments, the antibody or antigen-binding fragment thereof comprises a HCVR comprising the amino acid sequence of SEQ ID NO: 63, and a LCVR comprising the aminoi acid sequence of SEQ ID NO: 67. In some embodiments, the epitope comprises the amino acid sequence of SEQ ID NO: 29.

In one aspect, this disclosure relates to a pharmaceutical composition comprising an antibody or antigen-binding fragment thereof of this disclosure, and a pharmaceutically acceptable carrier or diluent.

In one aspect, this disclosure relates to a unit dosage form comprising a container containing an antibody or antigen-binding fragment thereof of any one of the antibodies of this disclosure. In some embodiments, the container is a vial, a syringe, a prefilled syringe, or an autoinjector.

In one aspect, this disclosure relates to a kit comprising a container containing an antibody or antigen-binding fragment thereof or any one of the antibodies of this disclosure, and instructions for use of the antibody or antigen-binding fragment thereof for the treatment of small cell lung cancer.

In one aspect, this disclosure relates to nucleic acid molecules encoding any one of the antibodies or antigen-binding fragments thereof of this disclosure. In some embodiments, this disclosure relates to a recombinant expression vector comprising the nucleic acid molecules.

In one aspect, this disclosure relates to a pair of nucleic acid molecules encoding heavy and light chain variable regions of any of the antibodies or antigen-binding fragments of this disclosure, wherein the pair of nucleic acid molecules comprises a first nucleic acid molecule encoding a heavy chain variable region of the antibody or antigen-binding fragment, and a second nucleic acid molecule encoding a light chain variable region of the antibody or antigen-binding fragment. In some embodiments, the pair of nucleic acid molecules are comprised within a recombinant expression vector.

In one aspect, this disclosure relates to an isolated host cell comprising any one of the antibodies or antigen-binding fragments of this disclosure, any one of the nucleic acid molecules of this disclosure, any one of the recombinant expression vectors of this disclosure, any pair of nucleic acid molecules of this disclosure, or any pair of recombinant expression vectors of this disclosure. In some embodiments, the isolated host cell is a mammalian host cell.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges and all values falling within such ranges are encompassed within the scope of the present disclosure. Each of the values discussed above or herein may be expressed with a variation of 1%, 5%, 10% or 20%. Other embodiments will become apparent from a review of the ensuing detailed description.

Other embodiments will become apparent from a review of the ensuing detailed description.

### BRIEF DESCRIPTION OF THE DRAWINGS

FIGS. 1A-1D illustrate upregulation of antigen-complexed autoantibodies in small cell lung cancer (SCLC). The mean coefficient signal of upregulated (p<0.05) IgG and IgM autoantibodies in SCLC are higher (one-way ANOVA) than any other cancer type analyzed on antibody-antigen complex arrays (FIG. 1A). N=98 SCLC and 125 controls; N=47 NSCLC and 47 controls; N=86 pancreatic cancer and 86 controls; N=139 colon cancer and 139 controls; N=121 breast cancers and 121 controls. FIGS. 1B-1D show that IgG and IgM autoantibodies are sequentially discovered in the Cardiovascular Health Study (CHS) pre-diagnostic cohort (1B), tested in the Fred Hutch diagnostic cohort (1C), and validated in a third cohort from Vanderbilt (1D). Expression in cases: ⋅ IgG increased; [symbol] IgM increased; − decreased; x insignificant.

FIGS. 2A-2B illustrate upregulation of antigens targeted by autoantibodies in SCLC. FIG. 2A shows representative immunohistochemical images of antigens targeted by peripheral autoantibodies on SCLC tissue microarrays (N=45-62 cases). Scale bar, 50 μM. FIG. 2B shows positive or negative scoring of tissue microarray cores. Positive scores are further categorized by stage at diagnosis.

FIG. 3 illustrates expression of tumor-associated antigens in some normal tissues. Representative normal tissue cores that demonstrate both positive and negative expression for each immunohistochemical stain as well as expression in normal lung cores are shown. Scale bar is 500 μM.

FIGS. 4A-4B illustrate that CD133 has cancer-specific glycosylation of sialyl-Lewis A (sLeA) in H82 and H69 SCLC cell lines. FIG. 4A shows immunoblots from H82 lysates immunoprecipitated (IP) with CA9, SPINT2, PLD3 or CD133 antibody and probed with sLeA antibody. Depleted (Dep), whole cell lysates (WCL) and target-specific IP are shown. Blue boxes indicate expected target protein size. FIG. 4B shows immunoblots from H69 lysates immunoprecipitated (IP) with CD133 antibody and probed with anti-CD133 or anti-sLeA antibodies. Depleted (Dep), whole cell lysates (WCL) and target-specific IP are shown. Boxes indicate expected target protein size.

FIGS. 5A-5C illustrate SCLC autoantibodies (AAbs) to CD133 target glycosylation motifs. FIG. 5A shows immunoblots from H82 cell lysates (Lys) immunoprecipitated (IP) with CD133 antibody or isotype control and probed with anti-CD133 and anti-sLeA antibodies. Dep is depleted cell lysate after IP. FIG. 5B shows immunoblots from H82 lysates immunoprecipitated (IP) with CD133 antibody after treatment with PRIME deglycosylase and probed with anti-CD133 and anti-sLeA antibodies. Whole cell lysates (WCL), control IP and target-specific IP are shown. FIG. 5C shows ELISA quantification of autoantibody levels from SCLC patient plasma that bound to CD133 from H82 cell lysates that had or had not been pre-treated with PRIME deglycosylase. (N=5/group) Error bars show SEM *p<0.05, **p<0.005, ***p<0.0005 with student's t test.

FIGS. 6A-6C illustrate that SCLC AAb can target isoaspartylated post-translational modifications. FIG. 6A shows immunoblots from GST-fusion proteins bound to glutathione beads, treated with or without isoaspartylation-inducing buffer (IsoAsp), incubated with plasma from SCLC patients and probed with anti-human IgG and anti-GST antibodies. Multiple experiments quantified are shown in FIG. 6B. FIG. 6C shows IgG from SCLC patient plasma bound to IsoAsp compared to wild type (WT) peptides that were measured by ELISA (N=8 cases).

FIGS. 7A-7B illustrate that TFRC is citrullinated. FIG. 7A shows immunoblots from H82 and H69 whole cell lysates (WCL) probed with antibodies to citrulline, CD133, TFRC, PLD3, SPINT2 and CA9. Boxes indicate expected target protein size. FIG. 7B shows immunoblots from H82 lysates immunoprecipitated (IP) with citrulline antibody and probed with CD133, PLD3, CA9 or TFRC antibody. CD133 and then PLD3 were used to sequentially probe the same blot. Boxes indicate expected target protein size.

FIGS. 8A-8D illustrate SCLC AAbs target citrullinated TFRC. FIG. 8A shows immunoblots from H82 or H69 whole cell lysates (WCL) probed with TFRC or citruilline antibodies. GAPDH was used for a loading control. FIG. 8B shows immunoblots from 3T3 or H82 WCL and H82 lysates immunoprecipitated (IP) with TFRC, citrulline (CIT) or isotype control antibody (CON) and probed with anti-TFRC or anti-citrulline antibodies. FIG. 8C shows IgG from SCLC patient plasma bound to citrullinated (Cit) compared to wild type (WT) peptides that were measured by ELISA (N=8). FIG. 8D shows IgG from 10 SCLC patients and 10 controls captured in complex with antigen (AAb-TAA) by anti-TFRC antibody, free from antigen (AAb) by TFRC recombinant protein, or by cit(PTM)/WT peptides and were quantified by ELISA. An anti-TFRC antibody was used as a positive control for TFRC recombinant protein. Error bars show SEM. P values were estimated by paired sample t tests.

FIGS. 9A-9B illustrate detection of SCLC by panel autoantibodies. In FIG. 9A, panel autoantibodies are analyzed as a function of the time of blood draw in the CHS (pre-diagnostic, 1-2 hr prior to diagnosis (dx) or <1yr to dx) and FH or VB (diagnostic, limited or extensive stage). *p=<0.05; **p=<0.005, ***p=<0.0005 unpaired t-test. In FIG. 9B, panel autoantibodies are significantly higher in plasma from SCLC cases (n=98) compared to cases from other cancers (N=75 NSCLC, N=86 pancreatic, N=87 colon) or controls (n=175) from all cohorts. **p=<0.001 unpaired t-test.

FIGS. 10A-10F illustrate a weak association of panel autoantibodies with smoking pack years. FIG. 10A shows that panel autoantibodies are not differentially expressed in current versus former smokers (current N=100, former N=140). In FIGS. 10B-10F, panel autoantibodies including PLD3 (FIG. 10B), TIMP2 (FIG. 10C), GRAP2 (FIG. 10D), SPINK1 (FIG. 10E), and TFRC (FIG. 10F) are loosely correlated with number of smoking pack years. Pearson correlation, N=240.

FIG. 11A shows that panel autoantibodies are not associated with chronic obstructive pulmonary disease (COPD) (N=19 controls and 31 cases).

FIG. 11B shows that panel autoantibodies are not associated with autoimmunity (type 1 diabetes (N=3), rheumatoid arthritis (N=2), hypothyroidism (N=2) or myasthenia gravis (N=2). One case had both type 1 diabetes and myasthenia gravis diagnoses (total N=44 controls and 8 cases).

FIGS. 12A-12C illustrate a risk prediction model that can accurately detect SCLC. Receiver operating characteristic (ROC) curve of the 5-autoantibody (AAb, dashed line) complex panel is identified by maximizing AUC in the Vanderbilt (VB) dataset (FIG. 12A), then fixing the coefficients and testing in the CHS cohorts (FIG. 12B) and Fred Hutch (FH) cohorts (FIG. 12C). ROC curves for pack years are indicated in dotted line, and the combination of pack years and 5-AAb panel are indicated in black solid line.

FIG. 13 shows the process for isolating, sequencing, and cloning SCLC-associated autoantibodies which bind to identified PTMs.

FIG. 14 shows the near-infrared imaging demonstrating that 770-labeled 2D2 antibody homes to H82 SCLC tumors in vivo.

FIG. 15 shows the chemiluminescent ELISA which demonstrates that the 2D2 antibody strongly binds to specific TFRC-cit peptide.

FIG. 16 illustrates that that commercial TFRC antibody made in rabbits recognizes TFRC in multiple normal human tissues and H82 SCLC cell line. However, the 2D2 antibody isolated from SCLC patient PBMCs recognizes only TFRC in SCLC and not TFRC in normal tissues.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims. Any embodiments or features of embodiments can be combined with one another, and such combinations are expressly encompassed within the scope of the present invention. Any specific value discussed above or herein may be combined with another related value discussed above or herein to recite a range with the values representing the upper and lower ends of the range, and such ranges are encompassed within the scope of the present disclosure.

Unless defined otherwise, all technical and scientific terms used herein have the same meaning as commonly understood by one of ordinary skill in the art to which this invention belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties. In case of conflict, the present specification, including explanations of terms, will control. In addition, the materials, methods, and examples are illustrative only and not intended to be limiting.

### Selected Abbreviations

SCLC: Small Cell Lung Cancer
NSCLC: Non-Small Cell Lung Cancer
WT: Wild Type
PTM: Post-translational Modification
ELISA: Enzyme-linked immunosorbent assay
IgG: Immunoglobulin G
IgM: Immunoglobulin M
AAb: Autoantibody
AAb-Ag: Autoantibody-antigen complex
TMA: Tissue Microarray
WCL: Whole cell lysate
PNS: Paraneoplastic Neurological Syndromes
CT: Computed Tomography
CIT: Citrulline
PCR: Polymerase Chain Reaction
DNA: Deoxyribonucleic acid
cfDNA: cell free DNA
IP: Immunoprecipitation
SNP: Single Nucleotide Polymorphism
sLeA: sialyl Lewis A

### Definitions

Administration: To provide or give a subject an agent by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes or any combination of techniques thereof.

The disclosed compositions or other therapeutic agents of the present disclosure can be formulated into therapeutically-active pharmaceutical compositions that can be administered to a subject parenterally or orally. Parenteral administration routes include, but are not limited to epidermal, intraarterial, intramuscular (IM, and depot IM), intraperitoneal (IP), intravenous (IV), intrasternal injection or infusion techniques, intranasal (inhalation), intrathecal, injection into the stomach, subcutaneous injections (subcutaneous (SQ and depot SQ), transdermal, topical, and ophthalmic. The disclosed compositions or other therapeutic agent can be mixed or combined with a suitable pharmaceutically acceptable excipients to prepare pharmaceutical compositions. Pharmaceutically acceptable excipients include, but are not limited to, alumina, aluminum stearate, buffers (such as phosphates), glycine, ion exchangers (such as to help control release of charged substances), lecithin, partial glyceride mixtures of saturated vegetable fatty acids, potassium sorbate, serum proteins (such as human serum albumin), sorbic acid, water, salts or electrolytes such as cellulose-based substances, colloidal silica, disodium hydrogen phosphate, magnesium trisilicate, polyacrylates, polyalkylene glycols, such as polyethylene glycol, polyethylene-polyoxypropylene-block polymers, polyvinyl pyrrolidone, potassium hydrogen phosphate, protamine sulfate, group 1 halide salts such as sodium chloride, sodium carboxymethylcellulose, waxes, wool fat, and zinc salts, for example. Liposomal suspensions may also be suitable as pharmaceutically acceptable carriers.

Upon mixing or addition of a disclosed composition, or other therapeutic agent, the resulting mixture may be a solid, solution, suspension, emulsion, or the like. These may be prepared according to methods known to those of ordinary skill in the art. The form of the resulting mixture depends upon a number of factors, including the intended mode of administration and the solubility of the agent in the selected carrier. Pharmaceutical carriers suitable for administration of the disclosed compositions or other therapeutic agent include any such carriers known to be suitable for the particular mode of administration. In addition, the disclosed composition or other therapeutic substance can also be mixed with other inactive or active materials that do not impair the desired action, or with materials that supplement the desired action, or have another action. Methods for solubilizing may be used where the agents exhibit insufficient solubility in a carrier. Such methods are known and include, but are not limited to, dissolution in aqueous sodium bicarbonate, using cosolvents such as dimethylsulfoxide (DMSO), and using surfactants such as TWEEN® (ICI Americas, Inc., Wilmington, Del.).

The disclosed compositions or other therapeutic agent can be prepared with carriers that protect them against rapid elimination from the body, such as coatings or time-release formulations. Such carriers include controlled release formulations, such as, but not limited to, microencapsulated delivery systems. The disclosed compositions or other therapeutic agent is included in the pharmaceutically acceptable carrier in an amount sufficient to exert a therapeutically useful effect, typically in an amount to avoid undesired side effects, on the treated subject. The therapeutically effective concentration may be determined empirically by testing the compounds in known in vitro and in vivo model systems for the treated condition. For example, an acceptable SCLC animal model may be used to determine effective amounts or concentrations that can then be translated to other subjects, such as humans, as known in the art.

Injectable solutions or suspensions can be formulated, using suitable non-toxic, parenterally-acceptable diluents or solvents, such as 1,3-butanediol, isotonic sodium chloride solution, mannitol, Ringer's solution, saline solution, or water; or suitable dispersing or wetting and suspending agents, such as sterile, bland, fixed oils, including synthetic mono- or diglycerides, and fatty acids, including oleic acid; a naturally occurring vegetable oil such as coconut oil, cottonseed oil, peanut oil, sesame oil, and the like; glycerine; polyethylene glycol; propylene glycol; or other synthetic solvent; antimicrobial agents such as benzyl alcohol and methyl parabens; antioxidants such as ascorbic acid and sodium bisulfite; buffers such as acetates, citrates, and phosphates; chelating agents such as ethylenediaminetetraacetic acid (EDTA); agents for the adjustment of tonicity such as sodium chloride and dextrose; and combinations thereof. Parenteral preparations can be enclosed in ampoules, disposable syringes, or multiple dose vials made of glass, plastic, or other suitable material. Buffers, preservatives, antioxidants, and the like can be incorporated as required. Where administered intravenously, suitable carriers include physiological saline, phosphate-buffered saline (PBS), and solutions containing thickening and solubilizing agents such as glucose, polyethylene glycol, polypropyleneglycol, and mixtures thereof. Liposomal suspensions, including tissue-targeted liposomes, may also be suitable as pharmaceutically acceptable carriers.

If a disclosed composition or other therapeutic agent is administered orally as a suspension, the pharmaceutical compositions can be prepared according to techniques well known in the art of pharmaceutical formulation and may contain a suspending agent, such as alginic acid or sodium alginate, bulking agent, such as microcrystalline cellulose, a viscosity enhancer, such as methylcellulose, and sweeteners/flavoring agents. Oral liquid preparations can contain conventional additives such as suspending agents, e.g., gelatin, glucose syrup, hydrogenated edible fats, methyl cellulose, sorbitol, and syrup; emulsifying agents, e.g., acacia, lecithin, or sorbitan monooleate; non-aqueous carriers (including edible oils), e.g., almond oil, fractionated coconut oil, oily esters such as glycerine, propylene glycol, or ethyl alcohol; preservatives such as methyl or propyl p-hydroxybenzoate or sorbic acid; and, if desired, conventional flavoring or coloring agents. When formulated as immediate release tablets, these compositions can contain dicalcium phosphate, lactose, magnesium stearate, microcrystalline cellulose, and starch and/or other binders, diluents, disintegrants, excipients, extenders, and lubricants.

If oral administration is desired, a disclosed composition, or other therapeutic substance can be provided in a composition that protects it from the acidic environment of the stomach. For example, a disclosed composition, or other therapeutic agent can be formulated with an enteric coating that maintains its integrity in the stomach and releases the active compound in the intestine. A disclosed composition or other therapeutic agent can also be formulated in combination with an antacid or other such ingredient. Oral compositions generally include an inert diluent or an edible carrier and can be compressed into tablets or enclosed in gelatin capsules. For the purpose of oral therapeutic administration, the disclosed composition or other therapeutic substance can be incorporated with excipients and used in the form of capsules, tablets, or troches. Pharmaceutically compatible adjuvant materials or binding agents can be included as part of the composition.

The capsules, pills, tablets, troches, and the like can contain any of the following ingredients or compounds of a similar nature: a binder such as, but not limited to, acacia, corn starch, gelatin, gum tragacanth, polyvinylpyrrolidone, or sorbitol; a filler such as calcium phosphate, glycine, lactose, microcrystalline cellulose, or starch; a disintegrating agent such as, but not limited to, alginic acid and corn starch; a lubricant such as, but not limited to, magnesium stearate, polyethylene glycol, silica, or talc; a gildant, such as, but not limited to, colloidal silicon dioxide; a sweetening agent such as sucrose or saccharin; disintegrants such as potato starch; dispersing or wetting agents such as sodium lauryl sulfate; and a flavoring agent such as peppermint, methyl salicylate, or fruit flavoring. When the dosage unit form is a capsule, it can contain, in addition to material of the above type, a liquid carrier, such as a fatty oil. In addition, dosage unit forms can contain various other materials that modify the physical form of the dosage unit, for example, coatings of sugar and other enteric agents. The disclosed composition or other therapeutic agent can also be administered as a component of an elixir, suspension, syrup, wafer, tea, chewing gum, or the like. A syrup may contain, in addition to the active compounds, sucrose or glycerin as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, they can be of the sustained release type so that the compounds need to be administered less frequently.

Agent: Any protein, nucleic acid molecule (including chemically modified nucleic acids), compound, antibody, small molecule, organic compound, inorganic compound, cell, such as T-cell, or other molecule of interest. Agent can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject, including treating a subject with or at-risk of acquiring SCLC).

In some examples, an agent can act directly or indirectly to alter the activity and/or expression of SCLC associated molecule, such as a SCLC early detection molecule. In a particular example, a therapeutic agent (such as an antisense compound or antibody) significantly alters the expression and/or activity of a SCLC associated molecule. An example of a therapeutic agent is one that can decrease the activity of a gene or gene product associated with SCLC, for example as measured by a clinical response (such as an increase survival time or a decrease in one or more signs or symptoms associated with SCLC). Therapeutically agents also include organic or other chemical compounds that mimic the effects of the therapeutically effective peptide, antibody, or nucleic acid molecule.

A “pharmaceutical agent” is a chemical compound or composition capable of inducing a desired therapeutic or prophylactic effect when administered to a subject, alone or in combination with another therapeutic agent(s) or pharmaceutically acceptable carriers. In a particular example, a pharmaceutical agent significantly reduces the expression and/or activity of a SCLC associated molecule thereby increasing a subject's survival time, reducing a sign or symptom associated with the disease, prolonging the onset of SCLC signs or symptoms.

Antibody: A polypeptide including at least a light chain or heavy chain immunoglobulin variable region which specifically recognizes and binds an epitope of an antigen, such as a SCLC associated molecule or a fragment thereof. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Antibodies of the present disclosure include those that are specific for a disclosed SCLC-associated molecule.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “VH”) and a heavy chain constant region (comprised of domains CH1, CH2 and CH3). Each light chain is comprised of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In certain embodiments of the disclosure, the FRs of the antibody (or antigen binding fragment thereof) may be identical to the human germline sequences, or may be naturally or artificially modified. An amino acid consensus sequence may be defined based on a side-by-side analysis of two or more CDRs.

The term antibody includes intact immunoglobulins, as well the variants and portions thereof, such as Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W. H. Freeman & Co., New York, 1997.

Typically, a naturally occurring immunoglobulin has heavy (H) chains and light (L) chains interconnected by disulfide bonds. There are two types of light chain, lambda (A) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. An antibody that binds RET will have a specific VH region and the VL region sequence, and thus specific CDR sequences. Antibodies with different specificities (such as different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a murine antibody that specifically binds a SCLC-associated molecule.

The present disclosure also includes antibodies comprising variants of any of the HCVR, LCVR, and/or CDR amino acid sequences disclosed herein having one or more conservative substitutions. For example, the present disclosure includes antibodies having HCVR and/or LCVR amino acid sequences with, e.g., 10 or fewer, 8 or fewer, 6 or fewer, 4 or fewer, etc. conservative amino acid substitutions relative to any of the HCVR or LCVR amino acid sequences disclosed herein. Also included are antibodies having at least 90%, at least 95%, at least 98% or at least 99% sequence identity to one of the HCVR and/or LCVR sequences discussed herein.

The term “recombinant”, as used herein, refers to antibodies or antigen-binding fragments thereof of the disclosure created, expressed, isolated or obtained by technologies or methods known in the art as recombinant DNA technology which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁸ M or less (e.g., a smaller Ko denotes a tighter binding). Methods for determining whether two molecules specifically bind are well known in the art and include, for example, equilibrium dialysis, surface plasmon resonance, and the like.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to the recited epitope.

An “isolated antibody”, as used herein, is intended to refer to an antibody that is substantially free of other antibodies (Abs) having different antigenic specificities.

Polynucleotides, as discussed herein, may encode all or a portion of an antibody or antigen-binding fragment as discussed throughout the present disclosure. In some cases, a single polynucleotide may encode both a HCVR and a LCVR (e.g., defined with reference to the CDRs contained within the respective amino acid sequence-defined HCVR and LCVR, defined with reference to the amino acid sequences of the CDRs of the HCVR and LCVR. respectively, or defined with reference to the amino acid sequences of the HCVR and LCVR, respectively) of an antibody or antigen-binding fragment, or the HCVR and LCVR may be encoded by separate polynucleotides (i.e., a pair of polynucleotides). In the latter case, in which the HCVR and LCVR are encoded by separate polynucleotides, the polynucleotides may be combined in a single vector or may be contained in separate vectors (i.e., a pair of vectors). In any case, a host cell used to express the polynucleotide(s) or vector(s) may contain the full complement of component parts to generate the antibody or antigen-binding fragment thereof. For example, a host cell may comprise separate vectors, each encoding a HCVR and a LCVR, respectively, of an antibody or antigen-binding fragment thereof as discussed above or herein. Similarly, the polynucleotide or polynucleotides, and the vector or vectors, may be used to express the full-length heavy chain and full-length light chain of an antibody as discussed above or herein. For example, a host cell may comprise a single vector with polynucleotides encoding both a heavy chain and a light chain of an antibody, or the host cell may comprise separate vectors with polynucleotides encoding, respectively, a heavy chain and a light chain of an antibody as discussed above or herein

The term “vector”, as used herein, means any molecule or particle used to deliver a foreign nucleic acid into a cell. For example, the nucleic a can be, for example, RNA or DNA. The molecule or particle can be, for example, a plasmid, cosmid, phage, or virus.

Autoantibody: An “autoantibody” is an antibody produced by the immune system that is directed against one or more of the individual's own proteins.

Epitope: The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

Alteration or modulation in expression: An alteration in expression of a gene, gene product or modulator thereof, such as one or more SCLC associated molecules disclosed herein, refers to a change or difference, such as an increase or decrease, in the level of the gene, gene product, or modulators thereof that is detectable in a biological sample (such as a sample from a subject at-risk or having SCLC) relative to a control (such as a sample from a subject without a SCLC) or a reference value known to be indicative of the level of the gene, gene product or modulator thereof in the absence of the disease. An “alteration” in expression includes an increase in expression (up-regulation) or a decrease in expression (down-regulation).

Array: An arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called antibody chips or biochips. The array of molecules makes it possible to carry out a very large number of analyses on a sample at one time. In certain example arrays, one or more molecules (such as an oligonucleotide probe or antibody) will occur on the array a plurality of times (such as twice), for instance to provide internal controls. The number of addressable locations on the array can vary, for example from at least two, to at least four, to at least 9, at least 10, at least 14, at least 15, at least 20, at least 30, at least 50, at least 75, at least 100, at least 150, at least 200, at least 300, at least 500, least 550, at least 600, at least 800, at least 1000, at least 10,000, or more. In a particular example, an array includes 2-100 addressable locations, such as 2-40 addressable locations. In particular examples, an array consists essentially of probes or primers or antibodies (such as those that permit amplification or detection) specific for SCLC as disclosed herein.

Binding or stable binding: An association between two substances or molecules, such as the hybridization of one nucleic acid molecule to another (or itself), the association of an antibody with a peptide, or the association of a protein with another protein or nucleic acid molecule. An oligonucleotide molecule binds or stably binds to a target nucleic acid molecule if a sufficient amount of the oligonucleotide molecule forms base pairs or is hybridized to its target nucleic acid molecule, to permit detection of that binding. “Preferentially binds” indicates that one molecule binds to another with high affinity, and binds to heterologous molecules at a low affinity.

Binding can be detected by any procedure known to one skilled in the art, such as by physical or functional properties of the target complex. For example, binding can be detected functionally by determining whether binding has an observable effect upon a biosynthetic process such as expression of a gene, DNA replication, transcription, translation, and the like. Methods of detecting binding of an antibody to a protein are disclosed herein and also can include known methods of protein detection, such as Western blotting.

Biological activity: The beneficial or adverse effects of an agent on living matter. When the agent is a complex chemical mixture, this activity is exerted by the substance's active ingredient or pharmacophore, but can be modified by the other constituents. Activity is generally dosage-dependent and it is not uncommon to have effects ranging from beneficial to adverse for one substance when going from low to high doses. In one example, the agent significantly reduces the biological activity of the one or more SCLC associated molecules disclosed herein which reduces one or more signs or symptoms associated with the SCLC.

Biomarker: Molecular, biological or physical attributes that characterize a physiological state and can be objectively measured to detect or define disease progression or predict or quantify therapeutic responses. For instance, a substance used as an indicator of a biologic state. It is a characteristic that is objectively measured and evaluated as an indicator of normal biologic processes, pathogenic processes, or pharmacologic responses to a therapeutic intervention. In one example, a biomarker is a protein or nucleic acid sequence of a corresponding gene that is indicator of SCLC.

Clinical outcome: Refers to the health status of a patient following treatment for a disease or disorder, such as SCLC, or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Contacting: Placement in direct physical association, including both a solid and liquid form. Contacting an agent with a cell can occur in vitro by adding the agent to isolated cells or in vivo by administering the agent to a subject.

Control: A sample or standard used for comparison with a test sample, such as a biological sample obtained from a patient (or plurality of patients) without a particular disease or condition, such as SCLC. In some embodiments, the control is a sample obtained from a healthy patient (or plurality of patients) (also referred to herein as a “normal” control), such as a normal biological sample or from a non-cancerous biological sample from the patient that has particular disease or condition, such as SCLC. In some embodiments, the control is a historical control or standard value (e.g., a previously tested control sample or group of samples that represent baseline or normal values (e.g., expression values), such as baseline or normal values of a particular gene, gene product in a subject without SCLC). In some examples, the control is a standard value representing the average value (or average range of values) obtained from a plurality of patient samples (such as an average value or range of values of the gene or gene products in the subjects without SCLC).

Detecting: Identifying the presence, absence or relative or absolute amount of the object to be detected.

Diagnosis: The process of identifying a disease, such as SCLC, by its signs, symptoms and results of various tests. The conclusion reached through that process is also called “a diagnosis.” Forms of testing commonly performed include blood tests, medical imaging, urinalysis, and biopsy.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced. The expression of a nucleic acid molecule can be altered relative to a normal (wild type) nucleic acid molecule. Alterations in gene expression, such as differential expression, include but are not limited to: (1) overexpression; (2) underexpression; or (3) suppression of expression. Alternations in the expression of a nucleic acid molecule can be associated with, and in fact cause, a change in expression of the corresponding protein.

Protein expression can also be altered in some manner to be different from the expression of the protein in a normal (wild type) situation. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain is removed or added; (4) expression of an increased amount of the protein compared to a control or standard amount; (5) expression of a decreased amount of the protein compared to a control or standard amount; (6) alteration of the subcellular localization or targeting of the protein; (7) alteration of the temporally regulated expression of the protein (such that the protein is expressed when it normally would not be, or alternatively is not expressed when it normally would be); (8) alteration in stability of a protein through increased longevity in the time that the protein remains localized in a cell; (9) alteration of the localized (such as organ or tissue specific or subcellular localization) expression of the protein (such that the protein is not expressed where it would normally be expressed or is expressed where it normally would not be expressed); and (10) alteration in structure, stability or function of a protein through post-translational modifications (PTMs), each compared to a control or standard. Controls or standards for comparison to a sample, for the determination of differential expression, include samples believed to be normal (in that they are not altered for the desired characteristic, for example a sample from a subject who does not have SCLC) as well as laboratory values (e.g., range of values), even though possibly arbitrarily set, keeping in mind that such values can vary from laboratory to laboratory.

Cytotoxic agent: The term “cytotoxic agent” as used herein refers to a substance that inhibits or prevents the function of cells and/or causes destruction of cells. The term is intended to include radioactive isotopes (e.g., At211, I131, I125, Y90, Re186, Re188, Sm153, Bi212, P32 and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents described herein. A tumoricidal agent causes destruction of tumor cells.

Toxin: A “toxin” is any substance capable of having a detrimental effect on the growth or proliferation of a cell.

Measure: To detect, quantify or qualify the amount (including molar amount), concentration or mass of a physical entity or chemical composition either in absolute terms in the case of quantifying, or in terms relative to a comparable physical entity or chemical composition.

Sample (or biological sample): A biological specimen containing genomic DNA, RNA (including mRNA), protein, or combinations thereof, obtained from a subject. Examples include, but are not limited to, peripheral blood, plasma, serum, urine, saliva, tissue biopsy, surgical specimen, and autopsy material.

Screening: As used herein, “screening” refers to the process used to evaluate and identify candidate agents that can be used to identify SCLC, such as early stage SCLC. In some cases, screening involves contacting a candidate agent (such as an antibody, small molecule or cytokine) with SCLC cells and testing the effect of the agent on expression of SCLC associated molecules.

Expression of a microRNA can be quantified using any one of a number of techniques known in the art and described herein, such as by microarray analysis or by qRT-PCR.

Sensitivity: The percent of diseased individuals (e.g., individuals with SCLC) in which the biomarker of interest is detected (true positive number/total number of diseased×100). Non-diseased individuals diagnosed by the test as diseased are “false positives”. In some examples, sensitivity of an assay describes the ability of the assay to accurately predict whether one has SCLC using the disclosed SCLC associated molecules, as compared to another assay method. For example, a marker with a sensitivity of at least 60%, including 70%, 75%, 80%, 85%, 90%, 95% or greater sensitivity is one that is capable of accurately predicting SCLC.

Specificity: The percent of non-diseased individuals for which the biomarker of interest is not detected (true negative/total number without disease×100). Diseased individuals not detected by the assay are “false negatives.” Subjects who are not diseased and who test negative in the assay, are termed “true negatives.”

Signs or symptoms: Any subjective evidence of disease or of a subject's condition, e.g., such evidence as perceived by the subject; a noticeable change in a subject's condition indicative of some bodily or mental state. A “sign” is any abnormality indicative of disease, discoverable on examination or assessment of a subject. A sign is generally an objective indication of disease.

Small Cell Lung Cancer or Carcinoma: A type of highly malignant cancer within the lung. Compared to non-small cell carcinoma, small cell lung carcinoma has a shorter doubling time, higher growth fraction, and earlier development of metastases. Small-cell lung carcinoma usually presents in the central airways and infiltrates the submucosa leading to narrowing of bronchial airways. Common symptoms include cough, dyspnea, weight loss, and debility. Smoking is a significant risk factor. Over 70% of patients with small-cell lung carcinoma present with metastatic disease; common sites include liver, adrenals, bone, and brain. Due to its high grade neuroendocrine nature, small-cell carcinomas can produce ectopic hormones, including adrenocorticotropic hormone (ACTH) and anti-diuretic hormone (ADH). Ectopic production of large amounts of ADH leads to syndrome of inappropriate antidiuretic hormone hypersecretion (SIADH). Lambert-Eaton myasthenic syndrome (LEMS) is a well-known paraneoplastic condition linked to small-cell carcinoma. SCLC is also referred to as “oat cell carcinoma” due to the flat cell shape and scanty cytoplasm.

SCLC is thought to originate from neuroendocrine cells (APUD cells) in the bronchus called Feyrter cells. Hence, they express a variety of neuroendocrine markers, and may lead to ectopic production of hormones like ADH and ACTH that may result in paraneoplastic syndromes and Cushing's syndrome. Approximately half of all individuals diagnosed with Lambert-Eaton myasthenic syndrome (LEMS) will eventually be found to have a small-cell carcinoma of the lung. Combined small-cell lung carcinoma can occur in combination with a wide variety of other histological variants of lung cancer, including extremely complex malignant tissue admixtures. When it is found with one or more differentiated forms of lung cancer, such as squamous cell carcinoma or adenocarcinoma, the malignant tumor is then diagnosed and classified as a combined small cell lung carcinoma (c-SCLC). C-SCLC is a recognized subtype of SCLC.

Subject: Living multi-cellular vertebrate organisms, a category that includes living multi-cellular vertebrate organisms, a category that includes both human and non-human mammals. In one embodiment, a subject is a patient with cancer (e.g., small cell lung cancer).

Tissue: A plurality of functionally related cells. A tissue can be a suspension, a semi-solid, or solid. Tissue includes cells collected from a subject, such as from the lung.

Treating a disease: A therapeutic intervention that ameliorates a sign or symptom of a disease or pathological condition related to a SCLC, such as a sign or symptom of SCLC. Treatment can induce remission or cure of a condition or slow progression, for example, in some instances can include inhibiting the full development of a disease, for example preventing development of a SCLC. Prevention of a disease does not require a total absence of disease. For example, a decrease of at least 10%, such as at least 20%, at least 25%, at least 30%, at least 40%, at least 50%, decrease in a sign or symptom associated with the condition or disease, such as SCLC, can be sufficient. As used herein, the term “ameliorating,” with reference to a disease or condition, refers to any observable beneficial effect of the treatment. The beneficial effect can be evidenced, for example, by a delayed onset of clinical symptoms of the disease or condition in a susceptible subject, a reduction in severity of some or all clinical symptoms of the disease or condition, a slower progression of the disease or condition, a reduction in the number of relapses of the disease or condition, an improvement in the overall health or well-being of the subject, by other parameters well known in the art that are specific to the particular disease or condition, and combinations of such factors.

PTPRU: An enzyme that is encoded by the PTPRU gene which is a member of the protein tyrosine phosphatase (PTP) family. PTPRU is also known as PTP-pi, PTP lambda, hPTP-J, PTPRO and PTP psi. PTPs are known to be signaling molecules that regulate a variety of cellular processes including cell growth, differentiation, mitotic cycle, and oncogenic transformation. This PTP possesses an extracellular region, a single transmembrane region, and two tandem intracellular catalytic tyrosine phosphatase domains, and thus represents a receptor-type PTP (RPTP). RPTPs are able to remove phosphate moieties from tyrosine residues.

TFRC (transferrin receptor): Transferrin receptor also known as Cluster of Differentiation 71 (CD71), is a protein that in humans is encoded by the TFRC gene.

Chimeric antigen receptor: The term “chimeric antigen receptor” (CAR) refers to molecules that combine a binding domain against a component present on the target cell, for example an antibody-based specificity for a desired antigen (e.g., a tumor antigen, such as BCMA) with a T cell receptor-activating intracellular domain to generate a chimeric protein that exhibits a specific anti-target cellular immune activity. Generally, CARs consist of an extracellular single chain antibody-binding domain (scFv) fused to the intracellular signaling domain of the T cell antigen receptor complex zeta chain, and have the ability, when expressed in T cells, to redirect antigen recognition based on the monoclonal antibody's specificity.

Polymerase Chain Reaction or PCR: As used herein, “PCR” refers to polymerase chain reaction which is a molecular biology technique used to amplify a single copy of a segment of DNA or RNA, generating thousands to millions of copies of a particular DNA or RNA sequence. PCR is commonly used to amplify the number of copies of a DNA or RNA segment for cloning or to be used in other analytical procedures.

Nucleic acid: The term “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cfDNA, cDNA, DNA-RNA hybrids, or a polymer comprising purine and pyrimidine bases, or other natural, chemically or biochemically modified, non-natural, or derivatized nucleotide bases. The backbone of the nucleic acid can comprise sugars and phosphate groups (as may typically be found in RNA or DNA), or modified or substituted sugar or phosphate groups.

Vector: A “vector,” as used herein, refers to a recombinant plasmid that comprises a nucleic acid to be delivered into a host cell, either in vitro or in vivo. The vector may be used as a vehicle to carry a foreign nucleic acid sequence, usually DNA, into another cell, where it can be replicated and/or expressed.

### Methods of Diagnosing and Treating Small Cell Lung Cancer (SCLC)

Aspects of the present disclosure provides methods for diagnosing and treating small cell lung cancer (SCLC) in a subject. The disclosed method includes detecting the presence of one or more autoantibodies that specifically bind one or more epitopes of one or more antigens in a biological sample obtained from the subject, wherein the one or more antigens is selected from the group consisting of CDH5, CD133, SPINK1, CDH23, NLRP7, TFRC, SPINT2, NADSYN1, HIF1A, GRAP2, MAPRE1, INHA, PTEN, CTSB, B3GNT6, PLD3, TIMP2, NUDT2, ANAPC2, GPLD1, PTPRU and CA9, and wherein the one or more epitopes of the antigen includes a post-translational modification (PTM). The disclosed method further includes diagnosing the subject with SCLC when the presence of the one or more autoantibodies is detected in the biological sample obtained from the subject. The disclosed method further comprises treating the SCLC in the subject by: (i) administering a chemotherapy regimen to the subject; (ii) administering an immunotherapy regimen to the subject; (iii) administering an antibody to the subject; (iv) performing surgical resection of the SCLC in the subject; and/or (v) contacting the subject with radiotherapy targeting the SCLC. As disclosed herein, the subject may include a human. In some embodiments, the subject may be a patient, such as patient suffering from or exhibiting one or more symptoms of SCLC or a patient at-risk of acquiring SCLC (e.g., who is a smoker or has a history of smoking). In some embodiments, the biological sample is serum or plasma obtained from the subject or patient. These patients or subjects may benefit from the methods of the present disclosure.

SCLC elicits the generation of autoantibodies that result in unique paraneoplastic neurological syndromes. The mechanistic basis for the formation of such autoantibodies is largely unknown but is key to understanding their etiology. Disclosed herein is a high-dimensional technique that enables detection of autoantibodies in complex with native antigens directly from patient plasma. In specific embodiments, this unique platform is used to screen 1,009 human plasma samples for 3,600 autoantibody-antigen complexes and it is found that SCLC plasma harbors, on an average, 4-fold higher disease-specific autoantibody signals compared to other cancers. Across multiple independent SCLC cohorts (N=240), a set of common but previously unknown autoantibodies are identified that are produced in response to both intra and extracellular tumor antigens. Several disease-specific post-translational modifications within extracellular proteins are further characterized that are targeted by these autoantibodies, including citrullination, isoaspartylation and cancer-specific glycosylation. Since most SCLC patients have metastatic disease at diagnosis, it is queried if these novel autoantibodies could be utilized for SCLC early detection and/or treatment. In embodiments, a risk-prediction model has been created using 5 autoantibodies with an average area under the curve of 0.84 that improved to 0.96 by incorporating cigarette smoke consumption in pack years. Taken together, the present disclosure provides a novel approach to identify circulating autoantibodies in SCLC with mechanistic insight into disease-specific immunogenicity and clinical utility.

In specific embodiments, the AAb-Ag complex capture platform takes advantage of endogenous humoral immunity to capture disease-relevant epitopes like PTMs in tumor specific antigens. By capturing AAbs bound to their immunogenic antigen, a set of AAbs may be identified and validated that has clinical utility in an early detection risk prediction model. Furthermore, in embodiments, a subset of AAbs may identify novel tumor cell surface antigens that have the potential to be targeted in a therapeutic setting. While most of the antigens identified in the present disclosure had not been previously described as relevant to SCLC, there were a couple of exceptions. For example, CD133 overexpression has been reported in SCLC stem cells (Sarvi, et al., 2014, Cancer Research 74, 1554-1565) and TFRC expression was higher in SCLC plasma derived microvesicles compared to controls (Pedersen, et al., 2022, Clinical Proteomics 19, 2). Interestingly, expression of antigen and production of AAb regardless of disease stage suggest that the AAb-identified tumor associated antigens are not lost to immunoediting. In addition to novel expression, the findings that AAbs target PTM epitopes adds to the growing body of evidence that tumor-specific AAbs predominantly recognize non-mutated self-proteins (Banville and Nelson, 2022, Cancer Cell 40, 356-358; Zaenker, et al., 2016, Autoimmunity Reviews 15, 477-483). In embodiments of the present disclosure, immunogenic PTMs to 3 tumor specific AAbs are identified (i.e., citrullination, isoaspartylation and cancer-specific glycosylation) based on screening for PTM modifications and sites. In autoimmunity, however, AAbs have been identified against many types of PTMs including phosphorylation, acetylation, hydroxylation, nitration and carbamylation (Doyle and Mamula, 2012, Current Opinion in Immunology 24, 112-118). These PTM-AAbs can make highly specific biomarkers.

AAbs are uniquely well suited for early detection biomarkers as they are obtained via a simple blood draw, produced in detectable quantities (despite low antigen concentration), upregulated years prior to clinical symptoms, and are highly antigen specific (Kobayashi, et al., 2020, Seminars in Immunology 47, 101388). One of the major reasons that SCLC early detection could prove successful is that there exists a clearly defined high-risk population: heavy cigarette smokers (Kenfield, et al., 2008, Tobacco Control 17, 198). As this uniform at-risk population is already eligible for low-dose CT lung cancer screening, AAbs recognizing unique molecular features of SCLC can be utilized to sort patients into high or low risk of malignancy categories during current protocols. An AAb high-risk result would flag a review or acquisition of a diagnostic CT while an AAb low-risk result would continue with annual lung cancer screening using LD-CT. There is also the potential of adding in additional factors to further define the highest at-risk patients for SCLC. AAbs to PNS antigens could pair well with the non-PNS AAbs disclosed here.

In particular embodiments, the 4-parameter risk prediction model generated in accordance with the present disclosure may use one or more antigens selected from the group consisting of SPINK1, GRAP2, TIMP2 and PLD3 or it may use one or more antigens selected from the group consisting of SPINT2, SPINK1, TIMP2 and TFRC. In particular embodiments, the 5-parameter risk prediction model generated in accordance with the present disclosure may use one or more antigens selected from the group consisting of SPINK1, TFRC, GRAP2, TIMP2 and PLD3. While the antibody array, in the illustrated examples below, contain over 3,600 antibodies to approximately 2,500 proteins that were selected for cancer relevance, other examples may show other SCLC-specific autoantigens that might also perform well with the disclosed antibody array. Furthermore, while the illustrated embodiments probed for only three types of PTMs that are cancer enriched, other embodiments may show other cancer-enriched PTMs. Additionally, it is to be noted that in some embodiments, the antibody-based nature of the disclosed methods can easily be converted to more high-throughput platforms such as multiple smaller arrays on a single slide or ELISAs, without departing from the scope of this disclosure.

Thus, the methods disclosed herein identify tumor-specific autoantigen-AAb complexes ex vivo. Isolating tumor targeted AAbs to their disease-specific antigens contributes to accurate detection of early-stage SCLC. Autoantibody production to non-PNS antigens are described herein and the SCLC-specific PTMs present in these antigens can cause AAb production to epitopes that would otherwise be suspected to be immune tolerant. The biomarker detection panel and the methods of diagnosing SCLC tumorigenesis, according to the present disclosure, may lead to implementation of early detection strategies and antibody-based therapeutics. In specific embodiments, the biomarker detection panel of the present disclosure may be capable of detecting SCLC in a subject or patient with a sensitivity and specificity of at least 60%, at least 70% or 75%, at least 80% or 85%, or at least 90% or 95%.

SCLC is one of the few malignancies which is considered as a “recalcitrant” cancer, underscoring the need for a greater understanding of the disease biology, more effective treatment strategies and earlier intervention. Current treatments in the management of SCLC includes chemotherapy, radiotherapy, surgical resection, immunotherapy, and/or a combination thereof. SCLC is very sensitive to chemotherapy and treatment usually induces rapid responses. The current first-line of treatment in SCLC (extensive stage) is platinum-based chemotherapy, four to six cycles of cis- or carboplatin plus etoposide (Mascaux, et al., 2000, Lung Cancer 30, 23-36). Carboplatin is generally preferred over cisplatin due to its similar efficacy and lower toxicity (Rossi, et al., 2012, J Clin Oncol 30, 1962). However, the majority of patients experience a relapse within the first year of treatment. In case of platinum-sensitive relapse, rechallenge with first-line chemotherapy is preferred (Frih, et al., 2013, Ann Oncol 24, Suppl. 6, vi99-vi105; Rudin, et al., 2015, J Clin Oncol 33, 4106-4111). Topotecan is the only drug that is formally approved as second-line of treatment for SCLC and remains the standard of care. Oral topotecan showed a response rate (RR) of 6-17% (O'Brien, et al., 2006, J Clin Oncol 24, 5441-5447). For limited stage SCLC, the standard treatment with curative intent consists of four cycles of platinum-doublet chemotherapy combined with radiotherapy, which improves overall survival compared with chemotherapy alone, even in elderly patients (Corso, et al., 2015, J Clin Oncol 33, 4240-4246). A concurrent approach is preferred and the timing of radiotherapy is important with the start of radiotherapy (45 Gy in 30 fractions twice-daily) preferably coinciding with the first or second cycle of chemotherapy (De Ruysscher, et al., 2006, J Clin Oncol 24, 1057-1063; De Ruysscher, et al., 2016, Ann Oncol 27, 1818-1828).

Surgery in SCLC may be considered for very small biopsy-proven tumors (very limited disease), cT1 NOMO, with confirmed negative mediastinal staging. Most commonly, a surgically removed lung nodule of unknown origin turns out to be a small SCLC. There is a tendency towards offering surgery for very small SCLC with negative lymph nodes, but concurrent chemoradiation is an alternative choice (Hiddinga, et al., 2021, Eur Respir Rev 30, 210079).

Furthermore, immunotherapy might play a role in the treatment of SCLC. The rationale for combining immunotherapy with chemotherapy in SCLC is the high mutational burden in this tumor, with potentially enhanced immunogenicity. Chemotherapy may stimulate the expression of tumoral antigens, priming the tumor for response to immunotherapy. In some examples, the immunotherapy regimen may comprise administering a patient with an antibody that binds one or more of the epitopes. As one example, the antibody may be a monoclonal antibody medication (e.g., ipilimumab, nivolumab) that activates the immune system by targeting CTLA-4 (Peters et al., 2020, Ann Oncol 31, LBA84). In some cases, the antibody may be conjugated to a cytotoxic agent (e.g., an antitumor or anticancer agent). In some embodiments, bispecific antibodies may be administered to patient that are capable of binding a target antigen and a T-cell antigen (e.g., CD3) for therapeutic purposes involving targeting T-cell immune responses to tissues/cells expressing the target antigen.

The immunotherapy regimen, in embodiments, may comprise a chimeric antigen receptor T-cell that specifically binds one or more of the epitopes. Chimeric antigen receptors (CARs) redirect T-cell specificity toward antibody-recognized antigens expressed on the surface of cells (e.g., cancer cells), while T-cell receptors extend the range of targets to include intracellular antigens (e.g., tumor antigens). One aspect of the present disclosure includes a CAR which is specific for antigens expressed on the surface of SCLC tumor cells. In some examples, a method of treating a patient diagnosed with SCLC may comprise removing immune effector cells or T-cells from a patient diagnosed with SCLC, genetically modifying said immune effector cells or T-cells with a vector comprising a nucleic acid encoding a chimeric antigen receptor, thereby producing a population of modified immune effector cells or T-cells, and administering the population of modified immune effector cells or T-cells to the same subject.

Circulating Autoantibodies in SCLC have Greater Sensitivity Compared to Other Cancers

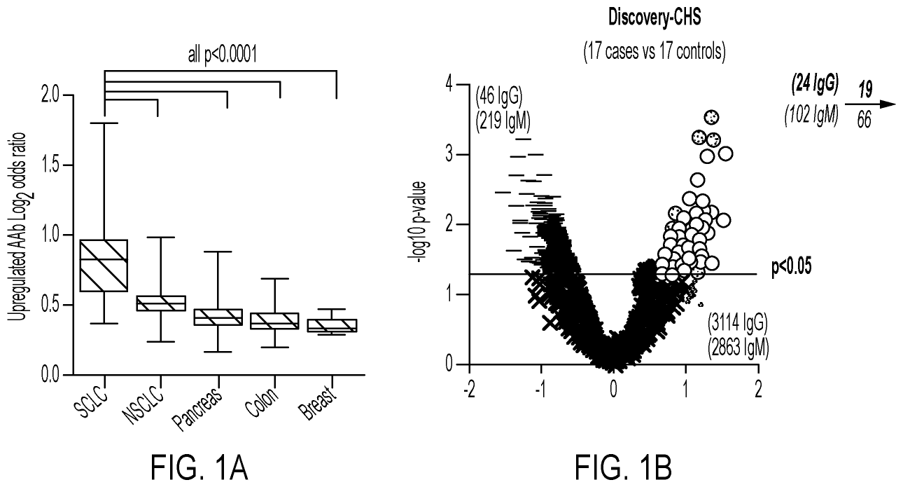

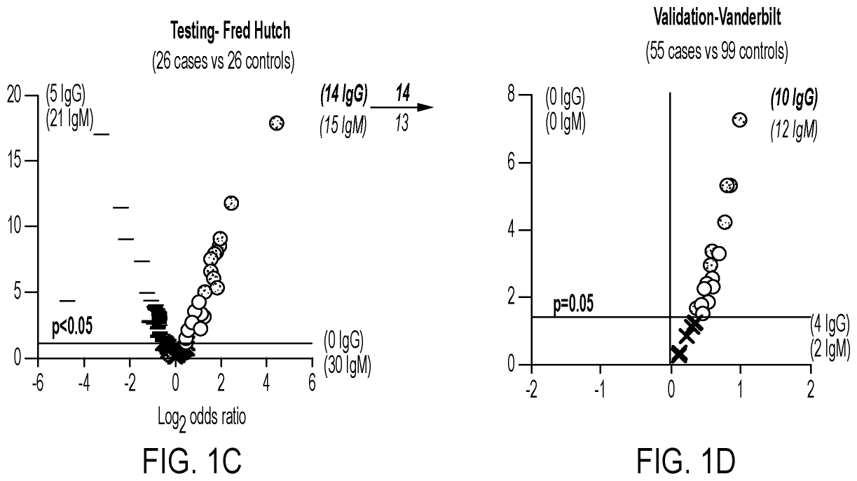

To demonstrate that cancer-specific AAbs were abundantly produced in SCLC, upregulated AAb-Ag content (i.e., ratio between cases and controls) were measured in a total of 1,009 plasma samples from cohorts of SCLC, non-small cell lung cancer (NSCLC), breast, colon and pancreas cancer. It was found that both cancer-specific IgG and IgM levels were significantly higher (p<0.0001) in SCLC and on average 4-fold higher than in other cancers (FIG. 1A). This indicated a greater production of disease-specific AAb in SCLC and underscored the potential of AAbs as SCLC-specific biomarkers. Next, specific AAbs consistently present across 3 independent cohorts of SCLC plasma were identified. First, 17 case-control pairs of pre-diagnostic plasma samples from the Cardiovascular Health Study (CHS, see Table 1) were analyzed for AAb-Ag complexes via an in-house discovery antibody array containing 3,600 antibody spots printed in triplicate (total 10,800 spots). It was observed that 46 IgG and 219 IgM significantly (p<0.05) downregulated and 24 IgG and 102 IgM significantly upregulated AAb-Ag complexes (FIG. 1B). The upregulated biomarkers were chosen and a novel in-house antibody array containing 19 of the 24 IgG and 66 of the 102 IgM capture antibodies for AAb-Ag complexes were designed. After hybridizing 26 case-control pairs from the Fred Hutch diagnostic cohort (Table 1), significant upregulation of 14 of the 19 IgG, (73.6% confirmation) and 15 of the 66 IgM (22.7% confirmation) AAb-Ag complexes were confirmed in cases compared to controls (FIG. 1C). To validate these results, 154 (55 SCLC cases and 99 matched controls) diagnostic plasma samples from Vanderbilt University (Table 1) were hybridized on a small in-house antibody array and all AAb-Ag complexes remained upregulated with 10/14 IgG (71.4% validation) and 12/13 IgM (92.3% validation) reaching statistical significance (FIG. 1D). Such a high rate of validation indicated that 22 promising SCLC-related AAb-Ag complexes were present for further investigation. A complete list of significantly upregulated and validated AAb-Ag complexes can be found in Table 2.

TABLE 1
Clinical characteristics of SCLC cohorts. Age and pack year are
averages in each group. Gender and smoking status are counts.

| Patient | CHS | | Fred Hutch | | Vanderbilt | |
|---|---|---|---|---|---|---|
| Characteristics | SCLC | Ctrl | SCLC | Ctrl | SCLC | Ctrl |
| Age (year) | 70.6 | 70.8 | 63.9 | 63.9 | 70.8 | 69.2 |
| Gender | | | | | | |
| Male | 7 | 7 | 18 | 18 | 32 | 65 |
| Female | 10 | 10 | 8 | 8 | 23 | 35 |
| Smoking Status | | | | | | |
| Current | 6 | 6 | 15 | 10 | 23 | 41 |
| Former | 11 | 11 | 11 | 16 | 32 | 59 |
| Never | 0 | 0 | 0 | 0 | 0 | 0 |
| Pack year | 42.3 | 30.8 | 53.3 | 42.8 | 54.9 | 60.4 |

TABLE 2
Targets of the upregulated AAb-Ag complexes validated
in 3 independent SCLC cohorts broken down by stage.

| Validated AAb-Ag Complex Targets | Target Entry Identifier |
|---|---|
| CDH5 | P33151 |
| CD133 | O43490 |
| SPINK1 | P00995 |
| CDH23 | Q9H251 |
| NLRP7 | Q8WX94 |
| TFRC | P02786 |
| SPINT2 | O43291 |
| NADSYN1 | Q6IA69 |
| HIF1A | Q16665 |
| GRAP2 | O75791 |
| MAPRE1 | Q15691 |
| INHA | P05111 |
| PTEN | P60484 |
| CTSB | P07858 |
| B3GNT6 | Q6ZMB0 |
| PLD3 | Q8IV08 |
| TIMP2 | P16035 |
| NUDT2 | P50583 |
| ANAPC2 | Q9UJX6 |
| GPLD1 | P80108 |
| PTPRU | Q92729 |
| CA9 | Q16790 |

### Autoantibodies Target Tumor-Associated Antigens in SCLC

It was hypothesized that the 22 validated AAbs were targeting tumor-associated antigens.

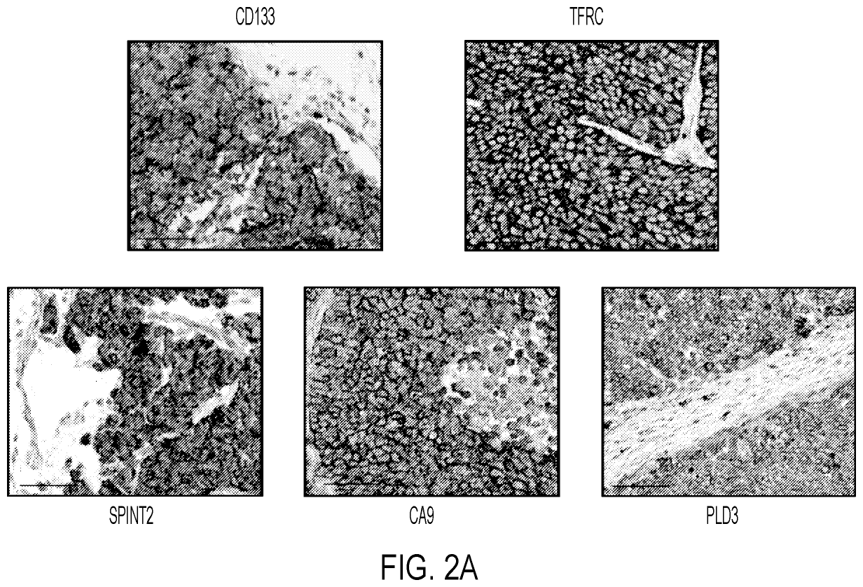

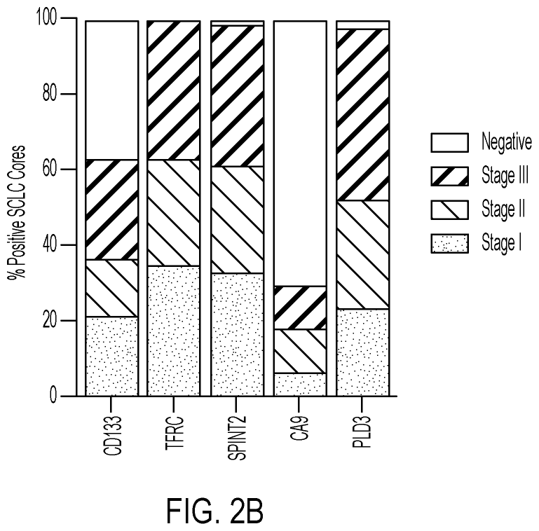

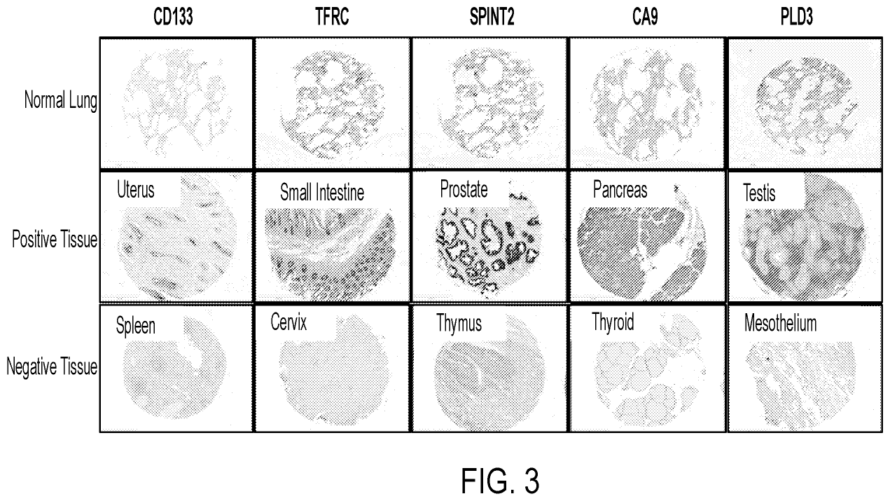

To test if this was indeed the case, 5 AAb antigens with transmembrane domains were prioritized that would likely be expressed on the cell surface of tumor cells and have potential translational relevance in future applications (e.g., imaging or antibody drug conjugates). Using antibodies for CD133, PLD3, TFRC, SPINT2 and CA9, immunohistochemistry was performed on SCLC tissue microarrays. CD133, PLD3, TFRC and SPINT2 showed high, homogenous expression in tumor cells, but not in adjacent stroma (FIG. 2A, staining controls in FIG. 3). In tumors that expressed CA9, most showed a focal staining pattern while approximately 20% displayed homogenous expression in tumor cells. Each core was scored as positive (>5% of tumor cells stained) or negative (<5% of tumor cells stained) (FIG. 2B) and it was found that >90% of tumor cores stained positive for TFRC, SPINT2 and PLD3, 60% for CD133 and 30% for CA9. Positive cores were further categorized into stage at diagnosis and demonstrated tumor-associated antigens were expressed with similar frequencies in stage I, II or III tumors. Cumulatively, this data validates the experimental approach utilizing AAbs to discover tumor-associated antigens that are expressed early and throughout tumorigenesis.

Despite SCLC having one the highest tumor mutational burdens, very few SCLC cell lines had non-silent mutations in the list of tumor-associated antigens: out of 51 SCLC cell lines with publicly available data, CD133 and CA9 were each mutated in 3, TFRC in 1 and SPINT2 and PLD3 had no SCLC cell lines with mutations (Table 3). This low rate of mutation (0-5.8%) suggested that the ‘neoantigens’ targeted by AAbs were likely occurring at the protein level. Since each of these proteins are exposed to the humoral immune system in normal tissues (FIG. 3), it was reasoned that the immune system was likely tolerant to their homeostatic expression. Thus, tumor-associated PTMs were investigated that could explain the observed production of AAbs.

TABLE 3
Autoantibody identified antigens are not commonly mutated in SCLC cell lines.

| | | Lineage | Variant | Variant | Reference | Protein | Cosmic |
|---|---|---|---|---|---|---|---|
| Gene | Cell Line | Subtype | Classification | Type | Allele | Change | Hs Cnt |
| TFRC | NCIH2286 | SCLC | Missense_Mutation | SNP | G | p.P500Q | 0 |
| CD133 | COLO668 | SCLC | Splice_Site | SNP | C | NA | 0 |
| CD133 | NCIH524 | SCLC | Missense_Mutation | SNP | A | p.F473L | 0 |
| CD133 | SW1271 | SCLC | Missense_Mutation | SNP | T | p.Y444F | 0 |
| CD133 | NCIH345 | SCLC | Silent | SNP | A | p.H581H | 0 |
| CD133 | MS1 | SCLC | Silent | SNP | G | p.G446G | 0 |
| CD133 | NCIH748 | SCLC | Silent | SNP | G | p.T461T | 0 |
| SPINT2 | None | | | | | | |
| CA9 | NCIH1105 | SCLC | Silent | SNP | C | p.A455A | 0 |
| CA9 | NCIH1048 | SCLC | Missense_Mutation | SNP | G | p.R178H | 0 |
| CA9 | NCIH2141 | SCLC | Missense_Mutation | SNP | G | p.W364L | 1 |
| CA9 | NCIH1688 | SCLC | Missense_Mutation | SNP | G | p.G55A | 0 |
| PLD3 | None | | | | | | |

SNP = Single Nucleotide Polymorphism.
Hs Cnt = Hotspot Count.

### Autoantibodies to CD133 Target Cancer-Specific Glycosylation Motif

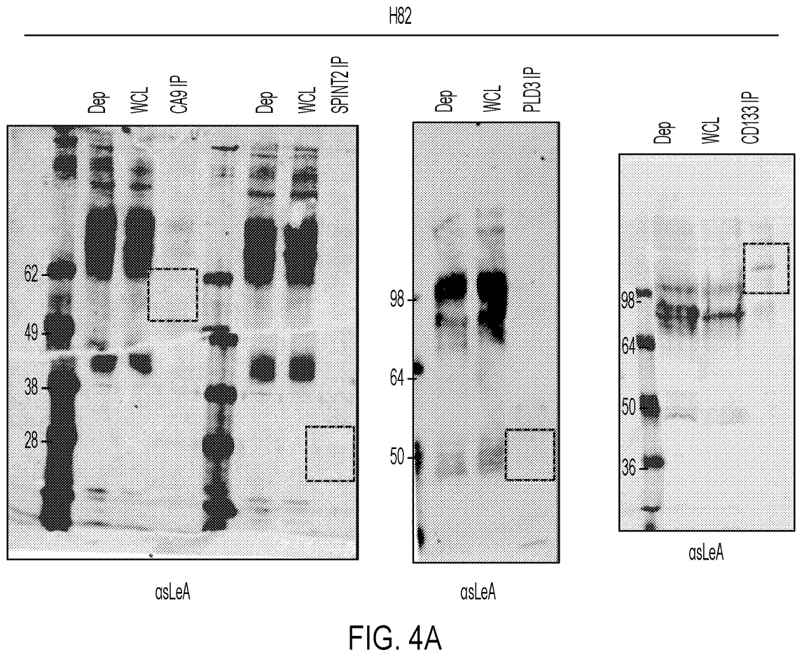

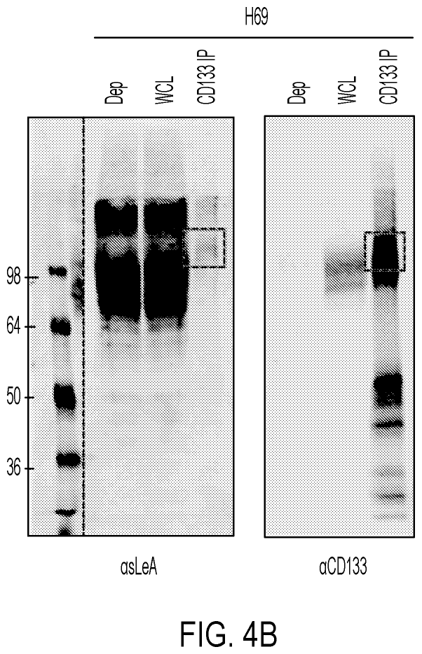

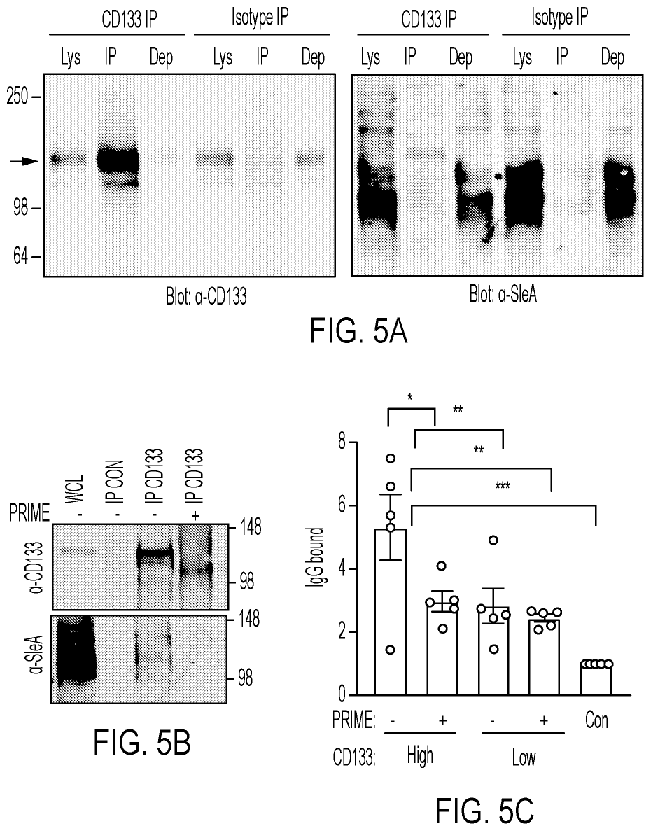

Tumor-associated glycans can induce autoantibody formation (Tikhonov, et al., 2020, Clinical Chemistry and Laboratory Medicine 58, 1611-1622) and most tumor biomarkers are glycoproteins (e.g., Prostate-specific antigen, CA125 (ovarian), CA19-9 (pancreas), α-fetoprotein (liver) and Carcinoembryonic antigen (colon)). To determine if AAb-identified antigens had tumor-specific glycan modifications, they were screened for the cancer-enriched carbohydrate antigen sialyl Lewis A (sLeA, also known as CA19-9) after immunoprecipitating target proteins from SCLC cell lines. In H82 cell lysates, sLeA appeared on multiple proteins, but only CD133 immunoprecipitation yielded a lone sLeA-positive, CD133-positive band (FIG. 4A). Immunoprecipitation of CD133 in H69, a second SCLC cell line, also showed a band positive for both SleA and CD133 (FIG. 4B). Additionally, this sLeA-positive, CD133-positive band was not present when an anti-mouse antibody (isotype control) was used for precipitation (FIG. 5A). To confirm glycosylation of CD133, it was immunoprecipitated from H82 cell lines and treated with PRIME deglycosylase to remove N-linked glycans and complex sialylated structures like sLeA. PRIME treatment collapsed the migration of CD133 confirming the removal of glycan residues and eliminated sLeA detection on CD133 protein (FIG. 5B). The specificity of autoantibodies present in SCLC patient plasma were assessed for glycan modified CD133 by creating a modified sandwich ELISA that replicated the array platform. A commercial CD133 antibody was plated to capture CD133 protein from H82 SCLC cell line lysates and, after incubation with plasma from SCLC patients with known high or low levels of CD133 AAbs (determined by array platform in FIG. 1C), IgG levels bound were verified to be significantly different by ELISA (FIG. 5C). Treatment of H82 lysates with PRIME deglycosylase diminished autoantibody signal to the same level as CD133-low plasma, arguing that glycan modifications were required for the increased autoantibody binding.

### Autoantibodies Bind to Isoaspartate Post-Translational Modifications in SPINK1

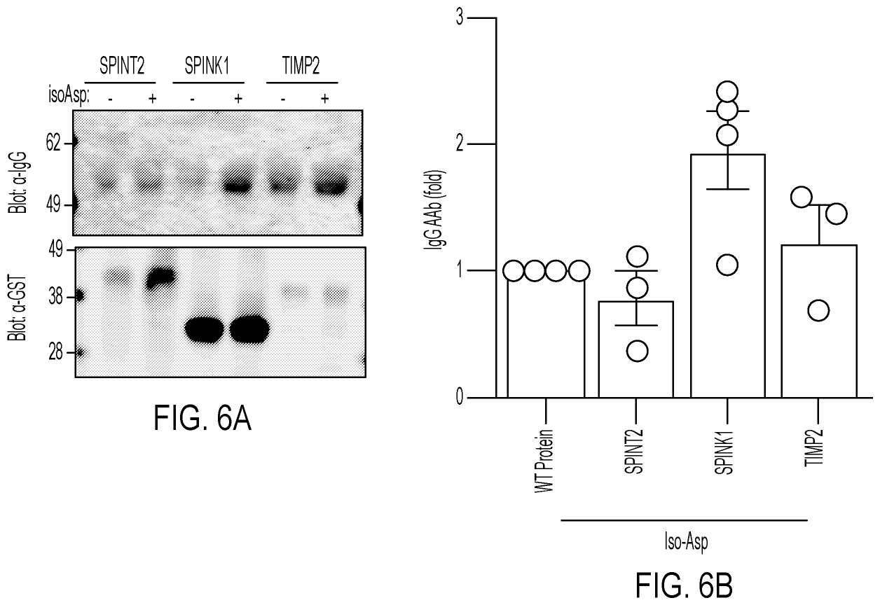

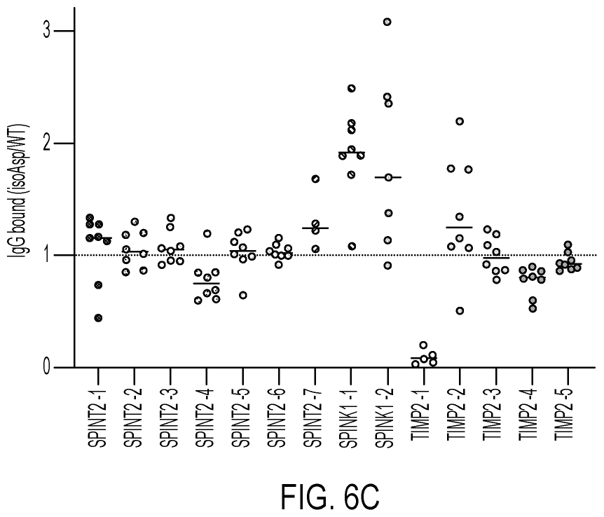

There has been one prior report that isoaspartate PTMs (IsoAsp) present in ELAVL4, a PNS antigen, can be immunogenic in SCLC-associated autoimmunity (Pulido, et al., 2016, Journal of Neuroimmunology 299, 70-78). IsoAsp is a disease-enriched, naturally occurring PTM that is formed by deamidation of asparagine or isomerization of aspartate. In order to look for IsoAsp-targeting AAbs, published in vitro methods were used to convert asparagine to IsoAsp residues in GST fusions of the extracellular regions of the smallest antigens (Geiger and Clarke, 1987, J Biol Chem 262, 785-794; Curnis, et al., 2006, Journal of Biological Chemistry 281, 36466-36476). The IsoAsp modified compared to unmodified proteins were used to pull down AAb from a pool of SCLC plasma, which qualitatively showed more IgG AAb bound to isoAsp SPINK1 but not isoAsp SPINT2 or TIMP2 by immunoblotting (FIG. 6A-6B). To determine specific sites of substitution, peptides were synthesized for all the asparagine residues in the extracellular regions of SPINT2 (6 sites), SPINK1 (2 sites), and TIMP2 (4 sites). Both wild type (WT) asparagine or isoAsp peptides were coated on assay plates, incubated with SCLC plasma samples, and AAb binding was detected using anti-human IgG (FIG. 6C). In agreement with the immunoblotting data, isoAsp was found at both positions in SPINK1 bound by at least 2-fold more AAbs than the corresponding WT peptide. Thus, this demonstrated that some AAbs in SCLC plasma recognize neoepitope PTM sites on SPINK1.

### SCLC AAbs Recognize Citrullinated TFRC not Present in Recombinant Protein

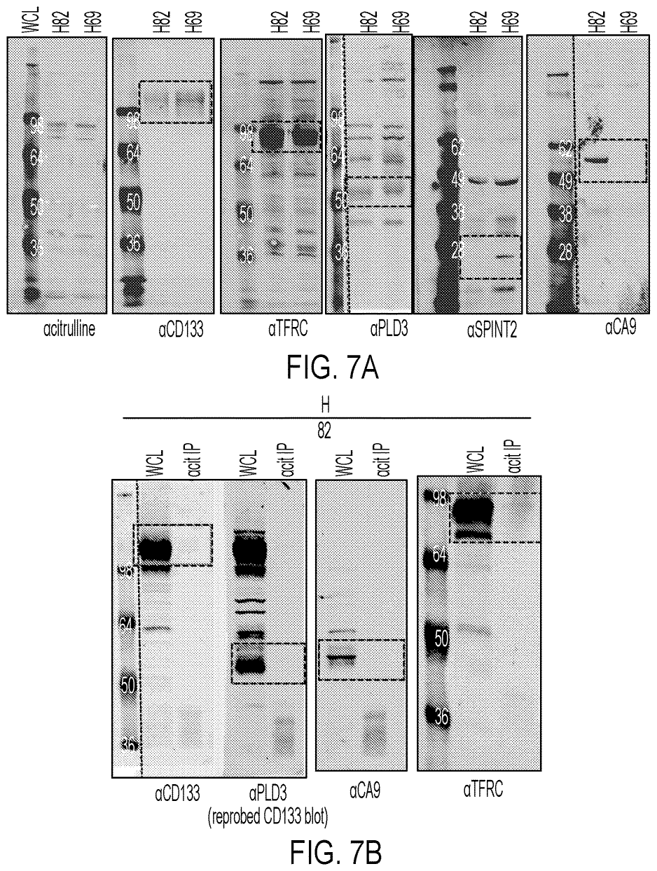

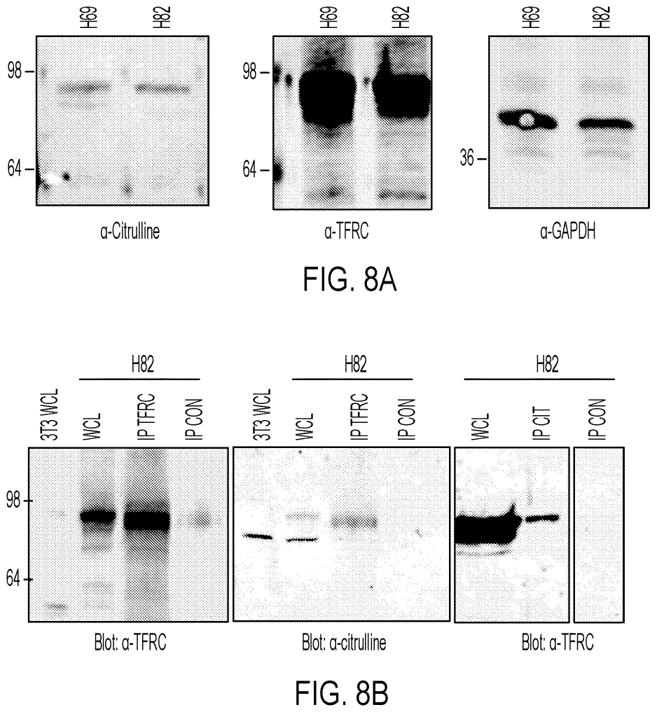

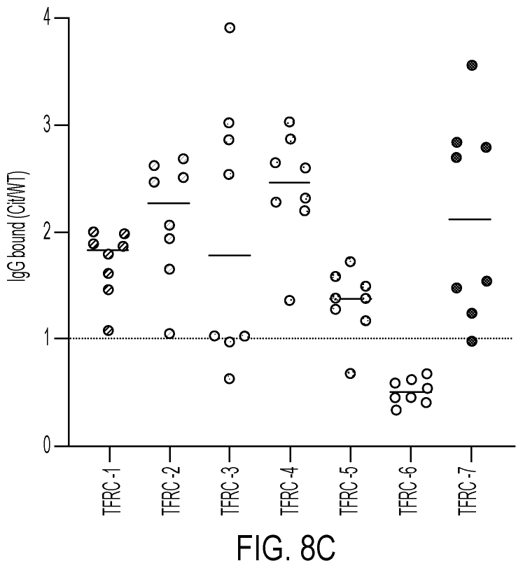

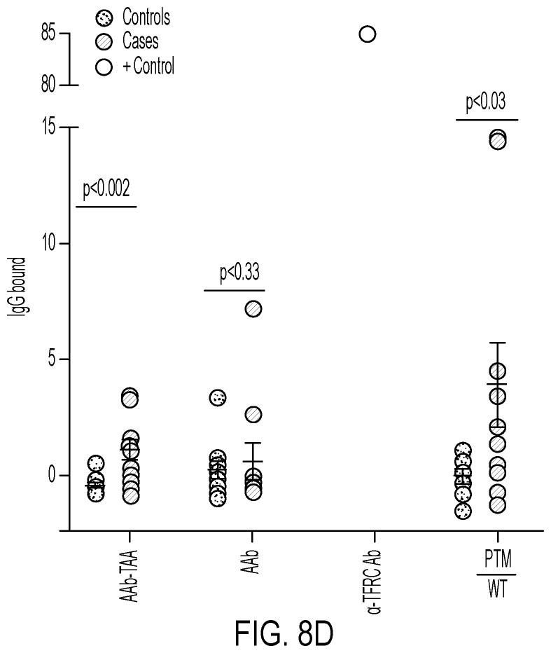

Citrulline, a unique amino acid generated by spontaneous arginine deamination has also been shown to be immunogenic in cancer but has yet to be described in SCLC (Brentville, et al., 2016, Cancer Res 76, 548-560; Pulido, et al., 2016, J Neuroimmunol 299, 70-78). Therefore, lysates from human SCLC H82 and H69 cells were screened with antibodies to citrulline, TFRC, PLD3, CD133, SPINT2 and CA9, and any similarly sized banding patterns overlapping between citrulline and target proteins were searched for. TFRC had potential overlapping bands with citrulline that were apparent at the expected protein size (FIG. 7A and FIG. 8A). To increase the sensitivity of citrulline detection, it was immunoprecipitated with a citrulline antibody and blotted back for target antibody expression and it was found that TFRC was the only target protein detected (FIG. 7B). These observations were expanded to show whole cell lysates from an immortalized fibroblast cell line 3T3 which had low levels of TFRC expression, but a corresponding band was not detected after probing with a citrulline antibody (FIG. 8B). Furthermore, immunoprecipitation with either anti-TFRC antibody and blotting back for citrulline or the reverse showed a matching band in H82 cells but none in the controls, indicating TFRC has arginine residues converted to citrulline specifically in H82 cells. The 32 arginines in the extracellular domain of TFRC was considered and published consensus motifs (Ju and Wang, 2018, Gene 664, 78-83) were used to choose 7 likely to undergo deamidation to citrulline. Peptides representing fragments of TFRC with either the wild-type arginine or citrullinated amino acid within the appropriate sequence were coated on assay plates and 5 of the 7 peptides with citrullinated residues pulled down more AAb from SCLC plasma than the corresponding WT peptide (FIG. 8C). TFRC AAbs, either complexed to antigen or free from antigen, were investigated in a subset of plasma samples from the Fred Hutch cohort. It was confirmed via ELISA that significantly (p<0.03) higher levels of AAb-TFRC complexes could be detected in SCLC plasma compared to controls. Conversely, no significant difference was detected in AAbs using TFRC recombinant protein as a capture antigen although an anti-TFRC antibody readily detected recombinant TFRC as a positive control (FIG. 8D). Finally, inclusion of 2 citrullinated TFRC peptides as capture antigens bound more AAbs from SCLC plasma compared to controls, suggesting that TFRC-AAbs were recognizing a citrullinated-neoantigen of TFRC not found on recombinant protein or arginine containing (wild type) peptides. Taken together, this data supports the AAb-Ag complex platform as a method to identify tumor-specific AAbs targeting post-translational ‘neoantigens’ that would have been difficult to detect using other approaches.

### Construction of a SCLC Risk-Prediction Model

Annual low dose computed tomography screening protocols currently in practice for early detection of NSCLC do not work for SCLC (Thomas, et al., 2018, Chest 154, 1284-1290). However, the clinical benefit of SCLC early detection is clear: when detected at limited stage, conventional chemotherapy is curative in nearly 20% of patients, and surgical resection, generally not considered, can be curative when combined with chemotherapy for very early stage patients (Lassen, et al., 1995, J Clin Oncol 13, 1215-1220; Winer, et al., 2011, Nature Medicine 17, 610-617). Having demonstrated that the AAbs produced in SCLC target specific PTMs in SCLC antigens, it was queried whether these unique AAbs could be utilized to generate a SCLC early detection risk prediction model. Therefore, all 22 validated AAb-Ag complexes were interrogated for their ability to reliably detect the presence of SCLC in plasma specimens. Since the Vanderbilt (VB) cohort had the largest sample size, this set was used as the training data and the best 4 or 5 marker combinations were calculated by maximizing AUC based on logistic regression. To account for class switching of IgM to IgG over the course of an immune response, both AAb isotypes were included for each marker. This identified a 4-marker panel with the highest overall AUC of 0.872 comprised of AAb against PLD3, TIMP2, GRAP2 and SPINK1 and, with the addition of TFRC, a 5-marker panel with an AUC=0.874 (Table 4). After fixing the coefficients using the data from the Vanderbilt set, the 5-marker risk prediction model was tested in the CHS cohort yielding an AUC=0.700 and the Fred Hutch (FH) cohort with an AUC=0.950.

TABLE 4
Maximizing AUC Analysis to Develop SCLC Risk
Prediction Model in VB Training Cohort.

| | AAb Targets | Training VB |
|---|---|---|
| 4 Markers | PLD3; TIMP2; GRAP2; SPINK1 | 0.872 |
| 5 Markers | PLD3; TIMP2; GRAP2; TFRC; SPINK1 | 0.874 |

A multiple logistic regression model was fit on VB data by including in the model the 5 AAb markers yielding the highest AUC. The prediction model was applied on CHS and FH testing datasets by partitioning cases and controls into high or low risk categories based on a positive or negative risk prediction score, respectively (Table 5). It was found that the risk prediction model performed well with positive predictive values of 76.9% in VB, 64.7% in CHS and 79.3% in FH and negative predictive values of 85.3% in VB, 64.7% in CHS and 86.9% in FH. When the pre-diagnostic samples from CHS were grouped by their time to diagnosis, there was higher accuracy in flagging high risk samples within a year prior to diagnosis (7/11, 63.6%) than 1-2 yrs prior to diagnosis (3/6, 50%). Moreover, in both the FH and VB cohorts, it was able to correctly identify 23/26 (88.5%) limited stage SCLC and 18/24 (75%) extensive stage SCLC in the high-risk pool.

TABLE 5
5-AAb panel risk prediction model can accurately sort patient
samples into high risk and low risk categories. Samples
were scored in each cohort as high or low risk based on
a positive or negative risk prediction score, respectively.

| | VB | | CHS | | FH | |
|---|---|---|---|---|---|---|
| | Control | Case | Control | Case | Control | Case |
| High Risk Score | 12 | 40 | 6 | 11 | 6 | 23 |
| Low Risk Score | 87 | 15 | 11 | 6 | 20 | 3 |

| | | | |
|---|---|---|---|
| Overall AUC | 0.874 | 0.700 | 0.950 |
| Overall Sensitivity | 0.727 | 0.647 | 0.885 |
| Overall Specificity | 0.879 | 0.647 | 0.679 |
| Sensitivity at 80% | 0.796 | 0.5 | 0.895 |
| Specificity | | | |

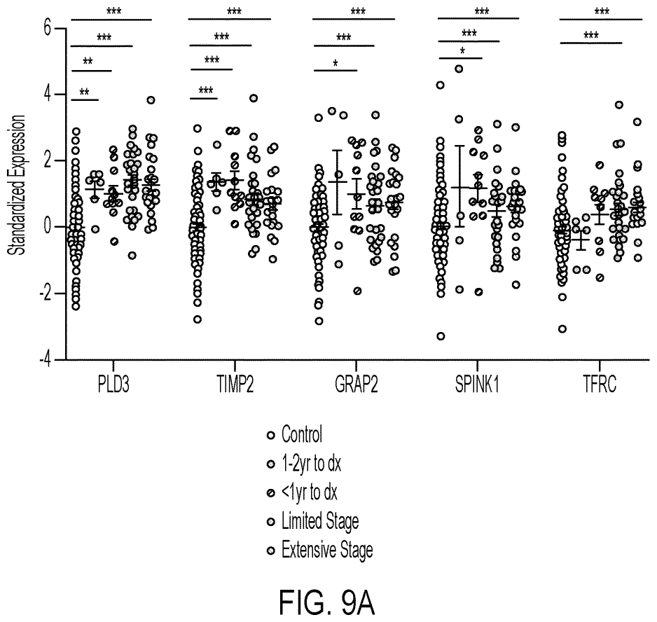

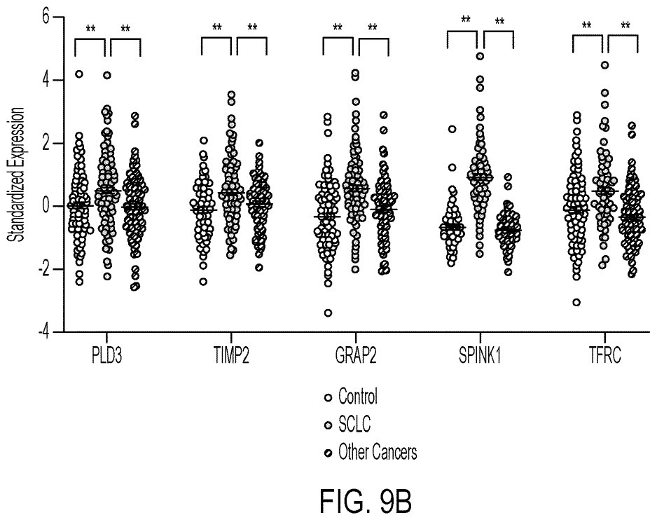

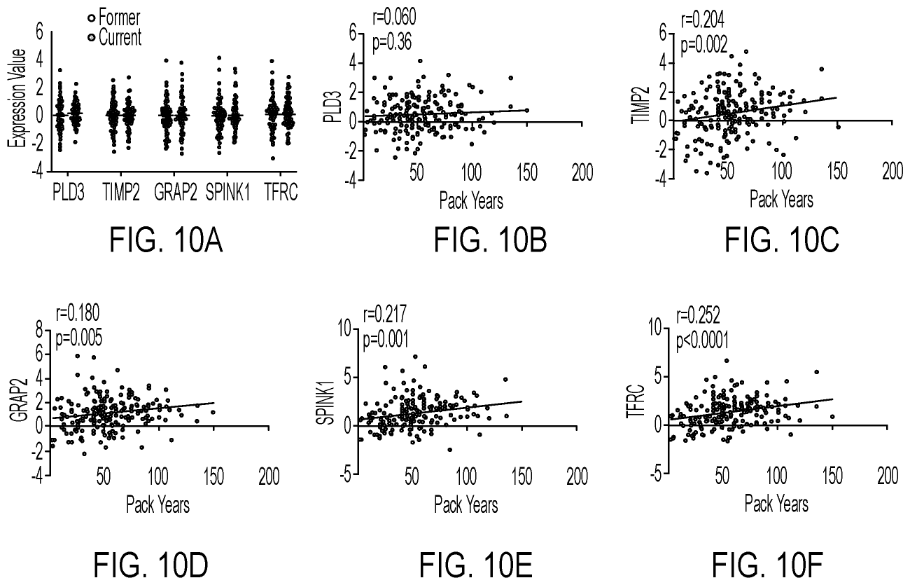

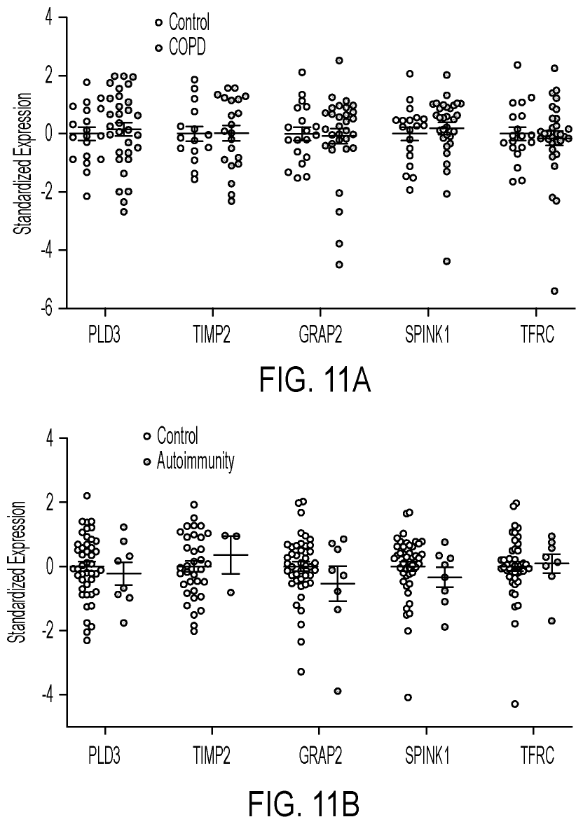

The 5 AAbs were investigated individually within each cohort in greater detail. When blood was drawn within the year prior to diagnosis, AAbs targeting PLD3, TIMP2, GRAP2, and SPINK1 were significantly upregulated (p=0.05), while AAbs to TFRC were trending up (p=0.15) (FIG. 9A). PLD3 and TIMP2 remained significantly upregulated (p<0.005) even up to two years prior to diagnosis. In diagnostic samples, all 5 panel members were highly significantly upregulated (p<0.0005) at both limited and extensive stage diseases. None of the panel markers were differentially expressed in current vs. former smoker cases (FIG. 10A) and PLD3 was not associated with smoking pack years (FIG. 10B). TIMP2, GRAP2, SPINK1 and TFRC were weakly associated (r<0.25) with smoking pack years (FIG. 10C-10F). Panel AAbs were not differentially expressed with chronic obstructive pulmonary disease (FIG. 11A) or autoimmunity (FIG. 11B) diagnoses. The individual performance of these markers was examined in plasma from other cancers including non-SCLC, pancreatic and colon, and it was found that none were significantly upregulated in the other diseases compared to controls or SCLC (FIG. 9B), suggesting SCLC specificity. Cumulatively, it suggests that these AAbs are specifically upregulated early in SCLC tumorigenesis and remain elevated throughout the disease process.

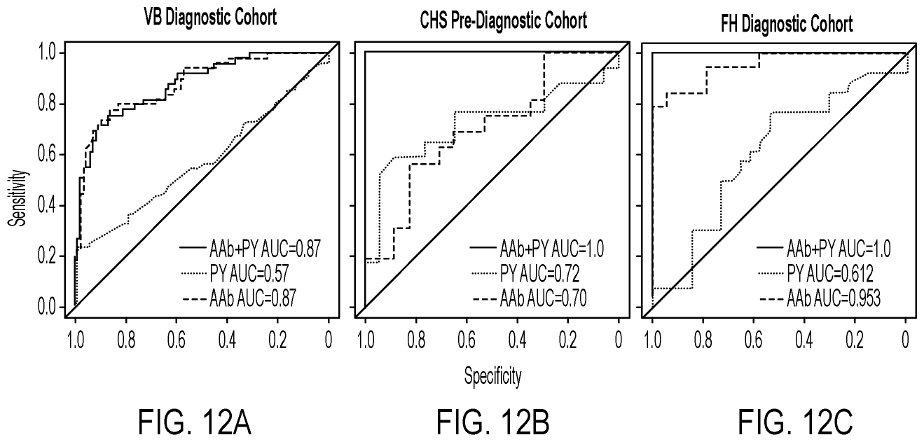

The single greatest risk factor for SCLC is current, heavy smoking, which can confer a 60 to 100-fold increase in the likelihood of developing SCLC for women and men, respectively (Wang, et al., 2017, Sci Rep 7, 1339). Since all three cohorts were matched on smoking history (current, former, never smokers), a pack year smoking history was incorporated into the 5-panel model. The continuous variable of smoking pack years alone had an AUC equal to 0.57 in VB, 0.72 in CHS and 0.612 in FH (FIG. 12A-12C). Combining smoking pack years with the AAb risk prediction model had minimal impact in the Vanderbilt cohort (AUC remained at 0.87), while the AUC increased to 1.0 in both the CHS and Fred Hutch cohorts.

### Experimental Methods and Materials

Additional details regarding the experiments discussed above are provided in the following paragraphs. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Study Design

All human plasma samples were obtained following institutional review board approval and informed consent. This study used three independent plasma cohorts: 1) The Cardiovascular Health Study (CHS) was a population-based, longitudinal study of coronary heart disease and stroke (Fried, et al., 1991, Ann Epidemiol 1, 263-276). Clinical examinations and plasma samples were completed annually for up to 10 years. 17 cases were newly diagnosed with SCLC within 24 months after a blood draw. These 17 cases were individually matched to controls based on age, gender, body mass index and smoking history. 2) Fred Hutchinson Early Detection and Prevention Clinic (Fred Hutch) for pulmonary nodule evaluation was comprised of N=26 SCLC patients and N=26 patients with benign pulmonary nodules (active IRB protocol no. 6663). Corresponding clinicopathological data was maintained in a highly-annotated database. The controls were matched to cases based on age, gender and smoking history. 3) 55 diagnostic plasma samples and 99 unmatched control plasma samples were matched on age, gender and smoking history and generously provided by Vanderbilt University Nashville, TN.

### AAb-Ag Complex Detection

The detection of AAb-Ag complexes using antibody arrays has been previously described (Winer, et al., 2011, Nature Medicine 17, 610-617). Briefly, antibody microarrays were printed in-house, incubated with patient plasma diluted 1:80, and probed with anti-human IgG-SeTau647 or IgM-DyLight550 antibody. Microarrays were imaged on a GenePix 4000B scanner and spot intensities measured in GenePix Pro 6.0 (Molecular Devices).

### Immunohistochemistry

Tissue microarrays (TMAs) containing 62 individual SCLC cases and 6 individual lung tissue controls (RLN681A, US Biolab) were stained for CD133 (#64326, 1:1000 Cell Signaling Technologies) or the same concentration of anti-rabbit IgG isotype antibody using the automated Leica Bond RX system and detected via Rabbit-HRP and DAB from Leica. The same TMAs were also used to stain for TFRC (HPA028598, 1:500 Sigma), SPINT2 (HPA011101, 1:200 Sigma), CA9 (HPA055207, 1:1000 Sigma) after heat induced epitope retrieval in citrate pH 6 buffer. Sections were probed with biotinylated pan-specific universal antibody for 10 minutes, streptavidin-HRP for 5 minutes (PK-8800, Vector Laboratories), and counterstained with hematoxylin with washes in between each step. After RLN681A was discontinued, a different TMA was purchased from US Biomax (BS04116a) containing 45 duplicate SCLC cores and 10 lung tissue controls and stained for PLD3 (HPA012800, 1:200 Sigma) using the Vector protocol described above. TMAs containing 33 normal tissues (3 individuals per organ, FDA999-1, US Biolab) were also stained for each of the antibodies as positive or negative controls. Stained slides were digitized using Leica Aperio whole slide scanner with 20× magnification. Scoring of TMA staining was performed in a blinded manner where each biopsy was determined to be either positive (>5% tumor cells stained) or negative (<5% tumor cells stained).

### ELISAs

Plate-based assays were developed to quantify autoantibody levels to different capture approaches. WT and PTM peptides were purchased from CH1 Scientific (sequences are listed in Table 6) and 10 μg of peptide was plated per well in maleimide 96 well plates (#15152, Pierce). White 96 well plates (#15042 Pierce) were coated with 1 μg/well of α-CD133 antibody (HB #7, Developmental Studies Hybridoma Bank) to capture CD133 from H82 lysates treated with or without PRIME glycosylase (#50-999-475, Fisher Scientific) at 1 U/50 μg cell lysate. α-TFRC antibody (HPA028598, Sigma) or TFRC recombinant protein (89-760aa, #11020-H07H, Sinobiological) was plated at 0.5 μg/well. After binding capture approach, all plates were washed and used as bait for autoantibodies from human plasma (1:500). All autoantibody signals were quantified using α-human IgG-HRP secondary (1:5000, #709-065-149, Jackson ImmunoResearch) and SuperSignal ELISA Femto (#37074, ThermoFisher) and a SpectroMax L Microplate reader (Molecular Devices).

TABLE 6
Peptide Sequences. Wildtype (WT) or post-translationally modified (PTM) peptide
sequences are listed below.

| Peptide | WT Sequence | PTM Sequence |
|---|---|---|
| SPINT2-1 | NVTDGS (SEQ ID NO: 1) | NVT{IsoAsp}GS (SEQ ID NO: 22) |
| SPINT2-2 | GGCDGNSN (SEQ ID NO: 2) | GGC{IsoAsp}GNSN (SEQ ID NO: 23) |
| SPINT2-3 | GGCDGNSN (SEQ ID NO: 2) | GGCDG{IsoAsp}SN (SEQ ID NO: 24) |
| SPINT2-4 | NAADSSV (SEQ ID NO: 3) | NAA{IsoAsp}SSV (SEQ ID NO: 25) |
| SPINT2-5 | GNKNSYR (SEQ ID NO: 4) | GNK{IsoAsp}SYR (SEQ ID NO: 26) |
| SPINT2-6 | RRQDSED (SEQ ID NO: 5) | RRQ{IsoAsp}SED (SEQ ID NO: 27) |
| SPINT2-7 | VERNSCN (SEQ ID NO: 6) | VER{isoAsp}SCN (SEQ ID NO: 28) |
| SPINK1-1 | YNELNGCTKI (SEQ ID NO: 7) | YNEL{iso-Asp}GCTKI (SEQ ID NO: 29) |
| SPINK1-2 | LCFENRKRQT (SEQ ID NO: 8) | LCFE{iso-Asp}RKRQT (SEQ ID NO: 30) |
| TIMP2-1 | TQKKSLNHRYQMG (SEQ ID NO: 9) | TQKKSL{iso-Asp}HRYQMG (SEQ ID NO: 31) |
| TIMP2-2 | VTEKNINGHQAKF (SEQ ID NO: 10) | VTEKNI{iso-Asp}GHQAKF (SEQ ID NO: 32) |
| TIMP2-3 | KAEGDGKMHI (SEQ ID NO: 11) | KAEG{iso-Asp}GKMHI (SEQ ID NO: 33) |
| TIMP2-4 | IKRSDGSCAW (SEQ ID NO: 12) | IKRS{iso-Asp}GSCAW (SEQ ID NO: 34) |
| TIMP2-5 | EKEVDSGNDI (SEQ ID NO: 13) | EKEV{iso-Asp}SGNDI (SEQ ID NO: 35) |
| TFRC-1 | DFPAARRLYW (SEQ ID NO: 14) | DFPAA{Cit}RLYW (SEQ ID NO: 36) |
| TFRC-2 | DFPAARRLYW (SEQ ID NO: 14) | DFPAAR{Cit}LYW (SEQ ID NO: 37) |
| TFRC-3 | NKVARAAA (SEQ ID NO: 15) | LYSA{Cit}GDF (SEQ ID NO: 38) |
| TFRC-4 | LNDRVMRVEY (SEQ ID NO: 16) | LNDRVM{Cit}VEYC (SEQ ID NO: 39) |
| TFRC-5 | LNDRVMRVEY (SEQ ID NO: 16) | LND{Cit}VMRVC (SEQ ID NO: 40) |
| TFRC-6 | LYSARGDFFRATS (SEQ ID NO: 17) | GDFF{Cit}ATS (SEQ ID NO: 41) |
| TFRC-7 | NKVARAAA (SEQ ID NO: 15) | NKVA{Cit}AAA (SEQ ID NO: 42) |
| CA9-1 | EEDSPR (SEQ ID NO: 18) | EE{IsoAsp}SPR (SEQ ID NO: 43) |
| CA9-2 | LRNNGHS (SEQ ID NO: 19) | LRN{IsoAsp}GHS (SEQ ID NO: 44) |
| CA9-3 | GVDSSPR (SEQ ID NO: 20) | GV{isoAsp}SSPR (SEQ ID NO: 45) |
| PLD3-1 | LRDNHTH (SEQ ID NO: 21) | LRD{isoAsp}HTH (SEQ ID NO: 46) |

### Immunoblotting

To induce isoaspartylation GST-fusion proteins were combined with magnetic glutathione beads (#78601, ThermoFisher) at 1:20 with NH₄HCO₃ (100 mM, pH 8.0) or PBS with 0.05% sodium azide and incubated at 37° C. for 48 h. After washing, treated GST-fusion proteins were incubated with human plasma at 1:10,000 at 4° C. overnight. Samples were directly lysed in Laemmli Sample Buffer containing 1× complete protease inhibitor cocktail, 1× phosphatase inhibitor cocktail and 2 mM phenylmethylsulfonyl fluoride (all from MilliporeSigma) and sonicated. Samples were separated by SDS-PAGE, blotted to nitrocellulose, checked for load with Ponceau S, blocked in 1% non-fat dry milk and probed with 1:5000 α-human IgG-AF680 (#709-625-149, Jackson ImmunoResearch) and 1:500 mouse α-GST (FHCC Antibody Technology Core) detected by α-mouse IgG-AF680 (1:10000, A32729, ThermoFisher). Immunoblots were imaged on an Odyssey CLx (LiCor; Lincoln, NE) with ImageStudio v5. CD133 protein was immunoprecipitated from H82 cells and treated with or without PRIME deglycosylase (#50-999-475, Fisher Scientific) at 1 U/50 μg cell lysate at 37° C. for 30 min and immunoblotted as described above with any differences noted below. Immunoblots were probed with antibodies to sLeA (1:2000, #10-C04E, Fitzgerald Industries) and CD133 (1:1000, #6436, Cell Signaling Technologies) and detected with α-mouse IgM-AlexaFluor680 and α-rabbit IgG-AlexaFluor790 (1:10000, #A10038, #A10043, ThermoFisher), respectively. Lysates from H82 or 3T3 cell lines were immunoprecipitated with 1 μg of αTFRC (#13-6800 Invitrogen), α-citrulline (ab100932, AbCam) or isotype control antibody (FHCC Antibody Technology Core) and protein G magnetic beads (#88847, ThermoFisher). Immunoblots were probed with antibodies to TFRC (1:1000, HPA028598, Sigma) or citrulline (1:500, MA5-27573, Invitrogen) and detected with α-mouse IgG-AlexaFluor680 and α-rabbit IgG-AlexaFluor790 (1:10000, A32729, A10043, ThermoFisher), respectively.

### Cell Culture

The NCI-H82 and NCI-H69 cell lines were acquired from ATCC, grown in Dulbecco's Modified Eagle Medium supplemented with 4 mM L-glutamine, 10% Fetal Bovine Serum and 1% Penicillin Streptomycin in an incubator at 37° C. and 5% CO₂ and routinely tested negative for mycoplasma by PCR.

### Statistical Analyses

Array data contain a format identical to two-channel gene expression arrays and analysis proceeds analogously as described previously (Rho and Lampe, 2013, J Proteome Res 12, 2311-2320). In GraphPad Prism, two groups were compared by t-test (type of t-test noted in brief description of the figures) and three or more groups were compared using one-way analysis of variance with Turkey's test. Logistic regression was used to identify the combination of multiple autoantibodies that best distinguished cases from controls. Receiver-Operator Characteristic curve was created and the Area Under the Curve was computed to evaluate the prediction capability. All related statistical analyses were performed using R statistical software (R Core Team (2021)).

### Identification of Autoantibodies

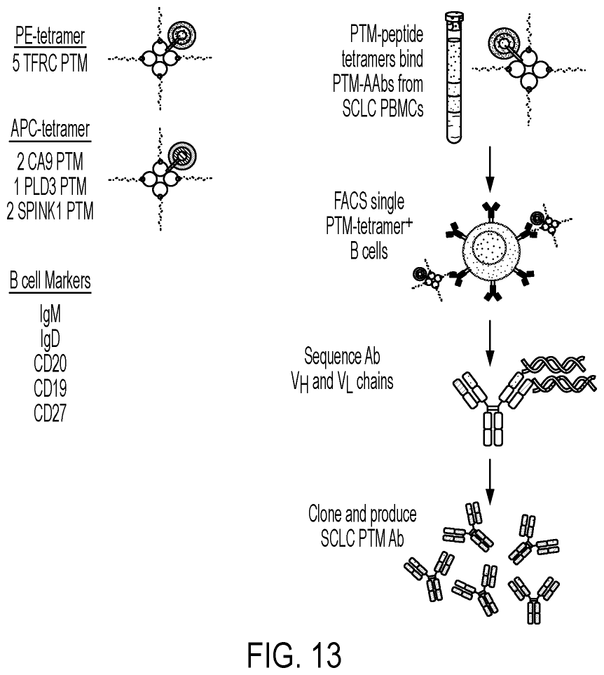

Once the immunogenic PTM peptides targeted by autoantibodies were identified, PTM-specific autoantibodies were isolated directly from SCLC patients and sequenced (FIG. 13). To accomplish this, PTM-binding B cells were enriched from SCLC patient peripheral blood mononuclear cells using PTM-peptide tetramers. PTM-peptide tetramers were created by combining PE- or APC-labeled magnetic tetramers containing streptavidin cores with biotinylated PTM peptides identified to bind more AAb from SCLC patient plasma compared to wildtype peptides. Five different PTM peptides were included in each PTM-tetramer; 5 citrullinated TFRC peptides were conjugated to PE-tetramers and 5 isoaspartylated peptides (3 CA9, 1 PLD3 and 1 SPINK1) were conjugated to APC-tetramers. These PTM-tetramers were then incubated with pooled peripheral blood mononuclear cells (PBMCs) from 3 different SCLC patients and tetramer bound cells were isolated with magnetic microbeads. This PTM-tetramer ‘enriched’ fraction was labeled with fluorescent antibodies and fluorescence activated cell sorted (FACS) into single cells. PTM-tetramer+ B cells were sorted, heavy and light chain antibody sequences from each B cell were amplified by RT-PCR, and heavy and light chain pairs were successfully sequenced. These sequences were cloned into human IgG expression vectors for antibody production and are shown in Table 7 with the location of the CRs underlined. The autoantigen PTM modifications to which the antibodies each bind are also shown in Table 7. The sequences of the CDRs were determined using the ImMunoGeneTics (IMGT) information system domain gap align. Table 8 lists the CDR sequences and their respective SEQ ID NOs.

TABLE 7
Antibody heavy and light change variable region sequences

| Antibody | Autoantigen | | |
|---|---|---|---|
| Name | Epitope | Heavy Chain Variable Region | Light Chain Variable Region |
| 2H8 | TFRC-5 | QVQLVESGGGVVQPGRSLRLSCAASGFT | EIVLTQSPATLFLFPGERVTFSCRASQSV |
| | | FSTYGMHWVRQAPGKGLQWVAQISNDG | NNYLAWYQQKPGQAPRLLIYDVSNRAT |
| | | SSKYYGDSVKGRFTISRDNSKNTLYLQM | GIPARFSGSGSGTDFTLTISSLDPEDFAV |
| | | NSLRVEDTGVYYCASSGSSGTWGQGTQ | YFCQQRSNWPLTFGGGTKVEIK (SEQ |
| | | VTVSS (SEQ ID NO: 47) | ID NO: 51) |
| 2H5 | TFRC-1 and | EVQLLESGGGLAQPGGSLRLSCGASGFT | DIQMTQSPSTLSASVGDRVTITCRASQSI |
| | TFRC-2 | FSSHALSWVRLAPGKGLEWVSAISGSGD | SSWLAWYQQKPGKAPNLLISKAFSVES |
| | | RTYYADSVKGRFTISRDNSKNMLYLQMN | GVPSTFSGSGSGTEFTLTISSLQPRDSA |
| | | SLRGEDTAVYYCAKEATESYATSWGQGT | SYYCQQYDSGWTFGQGTKVEIK (SEQ |
| | | LVTVSS (SEQ ID NO: 55) | ID NO: 59) |
| 1A6 | SPINK1-1 | QAGLLESGGGVVQPGRSLRLSCAASGFT | AIQLTQSPSSLSASVGDRVTITCRASQGI |
| | | FSSYGMHWVRQAPGKGLEWVAVIWYDG | SSYLAWYQQKPGKAPKLLMYGASTLQS |
| | | SNKYYADSVKGRFTISRDNSKNTLYLQM | GVPSRFSGSGSGTDFTLTISSLQPEDFA |
| | | NSLRAEDTAVYYCARGGGITGTVSRKQG | TYYCQQVNSYPLTFGGGTKVEIK (SEQ |
| | | MDVWGQGTLVTVSS (SEQ ID NO: 63) | ID NO: 67) |
| 1B3 | CA9-3 | QVQLVESGGGLVQPGGSLRLSCAASGFT | DIVMTQSPVTLAVSLGERATINCKSSQS |
| | | FSSYWMSWVRQAPGKGLEWVANIKQDG | VLYSSNNKNYLAWYQQKPGQPPKLLIY |
| | | SEEYYVDSVKGRFTISRDNAKSLLYLQMN | WASTRESGVPDRFSGSGSGTDFTLTISS |
| | | SLRPEDTAVYYCAGDSLPSSGWEGYFQ | LQAEDVAVYYCQQYYSSPRTFGQGTKV |
| | | HWGQGTLVTVSS (SEQ ID NO: 71) | EIK (SEQ ID NO: 75) |
| 2D2 | TFRC-4 | QVQLVESGGGVVQPGRSLRLSCAASGFT | DIQMTQSPLSLPVTPGEPASISCRSSQS |
| | | FNSFALMWVRQAPGKGLEWVAVISNDG | LLHADDGNTYLDWYLQKPGQSPQLLIYT |
| | | SNTYYADSVKGRFTISRDNSKNTLYLQM | LSSRASGVPDRFSGSGSGTNFTLRISRV |
| | | NSLRAEDTAVYYCARGSQYYGSGRYSR | EAEDVGVYYCMQRIESPLTFGGGTKVEI |
| | | KQFDYWGQGTMVTVSS (SEQ ID NO: 79) | K (SEQ ID NO:83) |

TABLE 8
Antibody Complementarity-determining regions (CDRs)

| Antibody | | | | | | |
|---|---|---|---|---|---|---|
| Name | HCDR1 | HCDR2 | HCDR3 | LCDR1 | LCDR2 | LCDR3 |
| 2H8 | GFTFSTYG | ISNDGSSK | ASSGSSGT | QSVNNY | DVS (SEQ | QQRSNWPLTF |
| | (SEQ ID NO: | (SEQ ID NO: | (SEQ ID NO: 50) | (SEQ ID NO: | ID NO: 53) | (SEQ ID NO: 54) |
| | 48) | 49) | | 52) | | |
| 2H5 | GFTFSSHA | ISGSGDRT | AKEATESYATS | QSISSW (SEQ | KAF (SEQ | QQYDSGWT |
| | (SEQ ID NO: | (SEQ ID NO: | (SEQ ID NO: 58) | ID NO: 60) | ID NO: 61) | (SEQ ID NO: 62) |
| | 56) | 57) | | | | |
| 1A6 | GFTFSSYG | IWYDGSNK | ARGGGITGTVS | QGISSY (SEQ | GAS (SEQ | QQVNSYPLT |
| | (SEQ ID NO: | (SEQ ID NO: | RKQGMDV | ID NO: 68) | ID NO: 69) | (SEQ ID NO: 70) |
| | 64) | 65) | (SEQ ID NO: 66) | | | |
| 1B3 | GFTFSSYW | IKQDGSEE | AGDSLPSSGWE | QSVLYSSNNK | WAS (SEQ | QQYYSSPRT |
| | (SEQ ID NO: | (SEQ ID NO: | GYFQH (SEQ ID | NY (SEQ ID | ID NO: 77) | (SEQ ID NO: 78) |
| | 72) | 73) | NO: 74) | NO: 76) | | |
| 2D2 | GFTFNSFA | ISNDGSNT | ARGSQYYGSG | QSLLHADDG | TLS (SEQ | MQRIESPLT |
| | (SEQ ID NO: | (SEQ ID NO: | RYSRKQFDY | NTY (SEQ ID | ID NO: 85) | (SEQ ID NO: 86) |
| | 80) | 81) | (SEQ ID NO: 82) | NO: 84) | | |

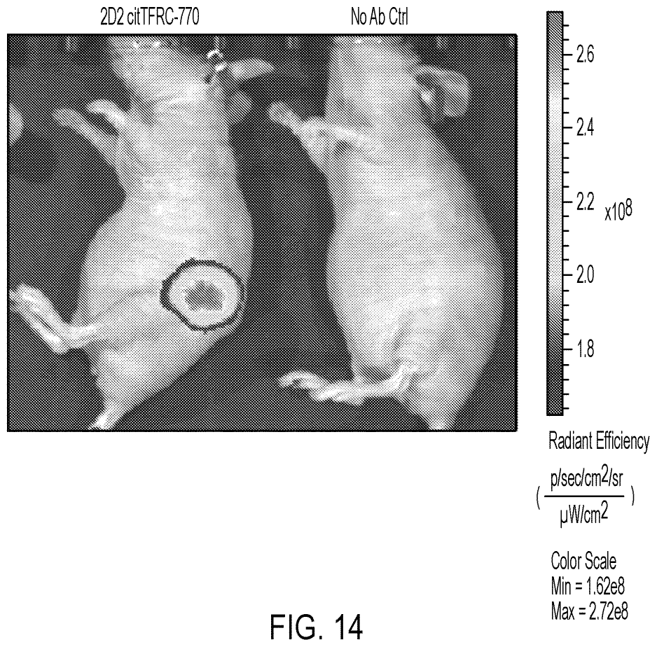

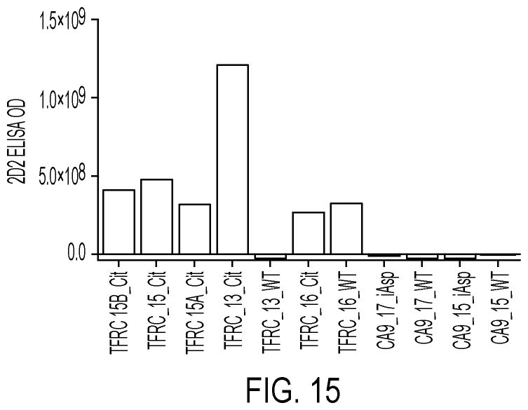

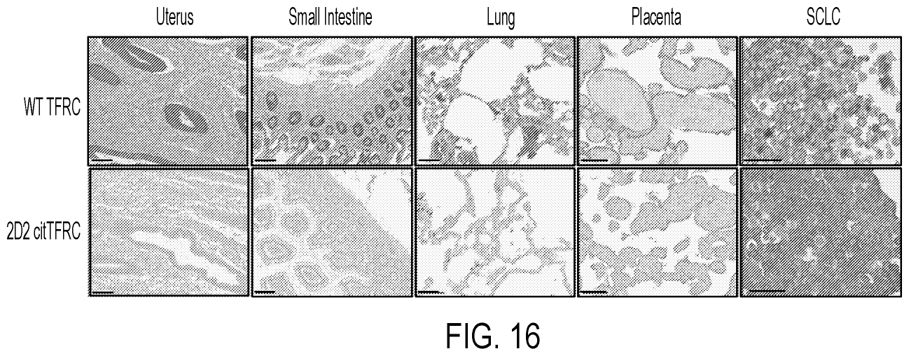

One PTM-specific antibody, 2D2, was isolated from SCLC PBMCs and further characterized. The 2D2 antibody has been successfully used to specifically image tumor TFRC in female athymic nude mice bearing subcutaneous flank H82 tumors (FIG. 14). To do this, the 2D2 antibody was labeled with CF770 and retro-orbitally injected mice. NIRF imaging was performed after 3, 4 and 5 days. Shown in FIG. 14 are the scans from day 5 with essentially all signal focused in the tumor. FIG. 15 shows that the 2D2 antibody predominantly binds to a specific TFRC sequence representing citrullinated peptide 13, to a lesser extent in other TFRC-citrullinated peptides but not to CA9, isoaspartylated or WT. These results indicates that the 2D2 antibody has specificity for both a specific TFRC sequence with citrulline in a specific location. FIG. 16 shows that the 2D2 antibody does not bind to TFRC in normal uterus, small intestine, lung or placenta while a commercial TFRC antibody (WT TFRC) binds extensively.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed is:

1. A method of treating small cell lung cancer (SCLC) in a subject, the method comprising:
(a) administering a chemotherapy regimen to the subject;
(b) administering an immunotherapy regimen to the subject;
(c) administering an antibody or antigen-binding fragment thereof to the subject;
(d) performing surgical resection of the SCLC in the subject; and/or
(e) contacting the subject with radiotherapy targeting the SCLC,
wherein the subject has autoantibodies that specifically bind one or more epitopes of one or more antigens selected from the group consisting of CDH5, CD133, SPINK1, CDH23, NLRP7, TFRC, SPINT2, NADSYN1, HIF1A, GRAP2, MAPRE1, INHA, PTEN, CTSB, B3GNT6, PLD3, TIMP2, NUDT2, ANAPC2, GPLD1, PTPRU and CA9, and wherein the one or more epitopes of the antigen comprises a post-translational modification (PTM).

2. The method of claim 1, wherein the subject has autoantibodies that specifically bind two or more epitopes of the one or more antigens.

3. The method of claim 1, wherein the subject has autoantibodies that specifically bind three or more epitopes of the one or more antigens.

4. The method of claim 1, wherein the subject has autoantibodies that specifically bind four or more epitopes of the one or more antigens.

5. The method of claim 1, wherein the subject has autoantibodies that specifically bind five or more epitopes of the one or more antigens.

6. The method of any one of claims 1-5, wherein the one or more antigens is selected from the group consisting of SPINK1, TFRC, GRAP2, TIMP2 and PLD3.

7. The method of any one of claims 1-5, wherein the one or more antigens is selected from the group consisting of SPINK1, GRAP2, TIMP2 and PLD3.

8. The method of any one of claims 1-5, wherein the one or more antigens is selected from the group consisting of SPINT2, SPINK1, TIMP2 and TFRC.

9. The method of any one of claims 1-8, wherein the one or more epitopes is selected from the group consisting of SEQ ID NOs: 1-21.

10. The method of any one of claims 1-9, wherein the PTM comprises citrullination, isoaspartylation and/or glycosylation.

11. The method of claim 10, wherein the PTM comprises citrullination and/or isoaspartylation.

12. The method of any one of claims 1-11, wherein the one or more epitopes is selected from the group consisting of SEQ ID NOs: 22-46.

13. A method of diagnosing and treating small cell lung cancer (SCLC) in a subject, the method comprising:
(a) detecting for presence of one or more autoantibodies that specifically bind one or more epitopes of one or more antigens in a biological sample obtained from the subject, wherein the one or more antigens is selected from the group consisting of CDH5, CD133, SPINK1, CDH23, NLRP7, TFRC, SPINT2, NADSYN1, HIF1A, GRAP2, MAPRE1, INHA, PTEN, CTSB, B3GNT6, PLD3, TIMP2, NUDT2, ANAPC2, GPLD1, PTPRU and CA9, and wherein the one or more epitopes of the antigen comprises a post-translational modification (PTM);
(b) diagnosing the subject with SCLC when the presence of the one or more autoantibodies is detected in the biological sample obtained from the subject; and
(c) treating the SCLC in the subject by:
(i) administering a chemotherapy regimen to the subject;
(ii) administering an immunotherapy regimen to the subject;
(iii) administering an antibody or antigen binding fragment thereof to the subject;
(iv) performing surgical resection of the SCLC in the subject; and/or
(v) contacting the subject with radiotherapy targeting the SCLC.

14. The method of claim 13, further comprising obtaining the biological sample from the subject.

15. The method of claim 13 or 14, wherein the biological samples is serum or plasma.

16. The method of any one of claims 13-15, wherein diagnosing the subject comprising diagnosing the subject with SCLC when the presence of autoantibodies that bind two or more epitopes of the one or more antigens is detected in the biological sample.

17. The method of any one of claims 13-15, wherein diagnosing the subject comprising diagnosing the subject with SCLC when the presence of autoantibodies that bind three or more epitopes of the one or more antigens is detected in the biological sample.

18. The method of any one of claims 13-15, wherein diagnosing the subject comprising diagnosing the subject with SCLC when the presence of autoantibodies that bind four or more epitopes of the one or more antigens is detected in the biological sample.

19. The method of any one of claims 13-15, wherein diagnosing the subject comprising diagnosing the subject with SCLC when the presence of autoantibodies that bind five or more epitopes of the one or more antigens is detected in the biological sample.

20. The method of any one of claims 13-19, wherein the one or more antigens is selected from the group consisting of SPINK1, TFRC, GRAP2, TIMP2 and PLD3.

21. The method of any one of claims 13-19, wherein the one or more antigens is selected from the group consisting of SPINK1, GRAP2, TIMP2 and PLD3.

22. The method of any one of claims 13-19, wherein the one or more antigens is selected from the group consisting of SPINT2, SPINK1, TIMP2 and TFRC.

23. The method of any one of claims 13-22, wherein the one or more epitopes is selected from the group consisting of SEQ ID NOs: 1-21.

24. The method of any one of claims 13-23, wherein the PTM comprises citrullination, isoaspartylation and/or glycosylation.

25. The method of claim 24, wherein the PTM comprises citrullination and/or isoaspartylation.

26. The method of any one of claims 13-25, wherein the one or more epitopes is selected from the group consisting of SEQ ID NOs: 22-46.

27. The method of any one of claims 1-26, wherein the subject is a human.

28. The method of any one of claims 1-27, wherein the chemotherapy regimen comprising one or more cycles of cisplatin or carboplatin with etoposide.

29. The method of any one of claims 1-28, wherein the immunotherapy regimen comprising a chimeric antigen receptor T cell that specifically binds one or more of the epitopes.

30. The method of any one of claims 1-28, wherein the immunotherapy regimen comprising an antibody that binds one or more of the epitopes.

31. The method of claim 30, wherein the antibody is conjugated to a cytotoxic agent.

32. The method of any one of claims 1-28, wherein the immunotherapy regimen comprising a bispecific antibody that binds one or more of the epitopes and a T-cell antigen.

33. The method of claim 32, wherein the T-cell antigen is CD3.

34. The method of any one of claims 1-33, wherein treating the SCLC in the subject is by administering an antibody or antigen binding fragment thereof to the subject.

35. The method of claim 34, wherein the antibody or the antigen binding fragment thereof specifically binds one or more epitopes of one or more antigens, wherein the one or more antigens is selected from the group consisting of CDH5, CD133, SPINK1, CDH23, NLRP7, TFRC, SPINT2, NADSYN1, HIF1A, GRAP2, MAPRE1, INHA, PTEN, CTSB, B3GNT6, PLD3, TIMP2, NUDT2, ANAPC2, GPLD1, PTPRU and CA9, and wherein the one or more epitopes of the antigen comprises a post-translational modification (PTM).

36. The method of claim 35, wherein the antibody or the antigen binding fragment thereof comprises three heavy chain complementarity determining regions (CDRs) contained within a heavy chain variable region (HCVR), and three light chain CDRs contained within a light chain variable region (LCVR), wherein the amino acid sequences of the HCVR/LCVR, respectively, comprise the amino acid sequences selected from the group consisting of SEQ ID NOs: 47/51, 55/59, 63/67, 71/75, and 79/83.

37. The method of claim 35, wherein the antibody or antigen-binding fragment comprises HCDR1-HCDR2-HCDR3-LCDR1-LCDR2-LCDR3 domains comprising the amino acid sequences, respectively, selected from the group consisting of SEQ ID NOs: 48-49-50-52-53-54, 56-57-58-60-61-62, 64-65-66-68-69-70, 72-73-74-76-77-78, and 80-81-82-84-85-86.

38. The method of claim 35, wherein the antibody or antigen-binding fragment thereof comprises a heavy chain variable region (HCVR) and a light chain variable region (LCVR), wherein the amino acid sequences of the HCVR/LCVR, respectively, are selected from the group consisting of SEQ ID NOs: 47/51, 55/59, 63/67, 71/75, and 79/83.

39. The method of any one of claims 34-38, wherein the antibody or antigen-binding fragment thereof binds to an epitope of TFRC-4 comprising a PTM, and wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 79, and three light chain CDRs (LCDR1, LCDR2, and LCDR3) contained within a light chain variable region (LCVR) comprising the amino acid sequence of SEQ ID NO: 83.

40. The method of claim 39, wherein the antibody or antigen-binding fragment comprises HCDR1-HCDR2-HCDR3-LCDR1-LCDR2-LCDR3 domains comprising the amino sequences of SEQ ID NOs: 80-81-82-84-85-86, respectively.

41. The method of claim 39 or 40, wherein the antibody or antigen-binding fragment thereof comprises a HCVR comprising the amino acid sequence of SEQ ID NO: 79, and a LCVR comprising the amino acid sequence of SEQ ID NO: 83.

42. The method of any one of claims 39-41, wherein the epitope comprises the amino acid sequence of SEQ ID NO: 39.

43. The method of any one of claims 34-38, wherein the antibody or antigen-binding fragment thereof binds to an epitope of CA9-3 comprising a PTM, and wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 71, and three light chain CDRs (LCDR1, LCDR2, and LCDR3) contained within a light chain variable region (LCVR) comprising the amino acid sequence of SEQ ID NO: 75.

44. The method of claim 43, wherein the antibody or antigen binding-fragment comprises HCDR1-HCDR2-HCDR3-LCDR1-LCDR2-LCDR3 domains comprising the amino sequences of SEQ ID NOs: 72-73-74-76-77-78, respectively.

45. The method of claim 43 or 44, wherein the antibody or antigen-binding fragment thereof comprises a HCVR comprising the amino acid sequence of SEQ ID NO: 71, and a LCVR comprising the amino acid sequence of SEQ ID NO: 75.

46. The method of any one of claims 43-44, wherein the epitope comprises the amino acid sequence of SEQ ID NO: 45.

47. The method of any one of claims 34-38, wherein the antibody or antigen binding fragment thereof binds to an epitope of TFRC-5 comprising a PTM, and wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 47, and three light chain CDRs (LCDR1, LCDR2, and LCDR3) contained within a light chain variable region (LCVR) comprising the amino acid sequence of SEQ ID NO: 51.

48. The method of claim 47, wherein the antibody or antigen binding-fragment comprises HCDR1-HCDR2-HCDR3-LCDR1-LCDR2-LCDR3 domains comprising the amino sequences of SEQ ID NOs: 48-49-50-52-53-54, respectively.

49. The method of claim 47 or 48, wherein the antibody or antigen-binding fragment thereof comprises a HCVR comprising the amino acid sequence of SEQ ID NO: 47, and a LCVR comprising the amino acid sequence of SEQ ID NO: 51.

50. The method of any one of claims 47-49, wherein the epitope comprises the amino acid sequence of SEQ ID NO: 40.

51. The method of any one of claims 34-38, wherein the antibody or antigen-binding fragment thereof binds to an epitope of TFRC-1 or TFRC-2 comprising a PTM, and wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 55, and three light chain CDRs (LCDR1, LCDR2, and LCDR3) contained within a light chain variable region (LCVR) comprising the amino acid sequence of SEQ ID NO: 59.

52. The method of claim 51, wherein the antibody or antigen-binding fragment comprises HCDR1-HCDR2-HCDR3-LCDR1-LCDR2-LCDR3 domains comprising the amino sequences of SEQ ID NOs: 56-57-58-60-61-62, respectively.

53. The method of claim 51 or 52, wherein the antibody or antigen-binding fragment thereof comprises a HCVR comprising the amino acid sequence of SEQ ID NO: 55, and a LCVR comprising the amino acid sequence of SEQ ID NO: 59.

54. The method of any one of claims 51-53, wherein the epitope comprises the amino acid sequence of SEQ ID NO: 36 or SEQ ID NO: 37.

55. The method of any one of claims 34-38, wherein the antibody or antigen-binding fragment thereof binds to an epitope of SPINK1-1 comprising a PTM, and wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 63, and three light chain CDRs (LCDR1, LCDR2, and LCDR3) contained within a light chain variable region (LCVR) comprising the amino acid sequence of SEQ ID NO: 67.

56. The method of claim 55, wherein the antibody or antigen-binding fragment comprises HCDR1-HCDR2-HCDR3-LCDR1-LCDR2-LCDR3 domains comprising the amino sequences of SEQ ID NOs: 64-65-66-68-69-70, respectively.

57. The method of claim 55 or 56, wherein the antibody or antigen-binding fragment thereof comprises a HCVR comprising the amino acid sequence of SEQ ID NO: 63, and a LCVR comprising the aminoi acid sequence of SEQ ID NO: 67.

58. The method of any one of claims 55-57, wherein the epitope comprises the amino acid sequence of SEQ ID NO: 29.

59. A biomarker detection panel for small cell lung cancer (SCLC), comprising one or more peptides comprising an epitope of from 7 to 50 amino acids in length from one or more antigens selected from the group consisting of CDH5, CD133, SPINK1, CDH23, NLRP7, TFRC, SPINT2, NADSYN1, HIF1A, GRAP2, MAPRE1, INHA, PTEN, CTSB, B3GNT6, PLD3, TIMP2, NUDT2, ANAPC2, GPLD1, PTPRU and CA9, wherein the epitope comprises a post-translational modification (PTM) bound by an autoantibody.

60. The biomarker detection panel of claim 59, wherein the panel is capable of detecting SCLC in a subject with a sensitivity and specificity of at least 60%.

61. The biomarker detection panel of claim 60, wherein the panel is capable of detecting SCLC in a subject with a sensitivity and specificity of at least 70% or 75%.

62. The biomarker detection panel of claim 60, wherein the panel is capable of detecting SCLC in a subject with a sensitivity and specificity of at least 80% or 85%.

63. The biomarker detection panel of claim 60, wherein the panel is capable of detecting SCLC in a subject with a sensitivity and specificity of at least 90% or 95%.

64. The biomarker detection panel of any one of claims 59-63, wherein the panel comprises two or more epitopes from the one or more antigens.

65. The biomarker detection panel of any one of claims 59-63, wherein the panel comprises three or more epitopes of the one or more antigens.

66. The biomarker detection panel of any one of claims 59-63, wherein the panel comprises four or more epitopes of the one or more antigens.

67. The biomarker detection panel of any one of claims 59-63, wherein the panel comprises five or more epitopes of the one or more antigens.

68. The biomarker detection panel of any one of claims 59-67, wherein the one or more antigens is selected from the group consisting of SPINK1, TFRC, GRAP2, TIMP2 and PLD3.

69. The biomarker detection panel of any one of claims 59-67, wherein the one or more antigens is selected from the group consisting of SPINK1, GRAP2, TIMP2 and PLD3.

70. The biomarker detection panel of any one of claims 59-67, wherein the one or more antigens is selected from the group consisting of SPINT2, SPINK1, TIMP2 and TFRC.

71. The biomarker detection panel of claim 70, wherein the one or more epitopes is selected from the group consisting of SEQ ID NOs: 1-21.

72. The biomarker detection panel of any one of claims 59-71, wherein the PTM comprises citrullination, isoaspartylation and/or glycosylation.

73. The biomarker detection panel of claim 72, wherein the PTM comprises citrullination and/or isoaspartylation.

74. The biomarker detection panel of claim 45, wherein the one or more epitopes is selected from the group consisting of SEQ ID NOs: 22-46.

75. The biomarker detection panel of any one of claims 34-49, wherein the panel is for detecting an autoantibody that binds the one or more epitopes of the one or more antigens in a biological sample obtained from a subject.

76. An isolated antibody or antigen binding fragment thereof that binds to an epitope of TFRC-4 comprising a PTM, wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 79, and three light chain CDRs (LCDR1, LCDR2, and LCDR3) contained within a light chain variable region (LCVR) comprising the amino acid sequence of SEQ ID NO: 83.

77. The antibody or antigen-binding fragment of claim 76, wherein the antibody or antigen binding fragment comprises HCDR1-HCDR2-HCDR3-LCDR1-LCDR2-LCDR3 domains comprising the amino sequences of SEQ ID NOs: 80-81-82-84-85-86, respectively.

78. The antibody or antigen-binding fragment of claim 76 or 77, wherein the antibody or antigen-binding fragment thereof comprises a HCVR comprising the amino acid sequence of SEQ ID NO: 79, and a LCVR comprising the amino acid sequence of SEQ ID NO: 83.

79. The antibody or antigen-binding fragment of any one of claims 76-79, wherein the epitope comprises the amino acid sequence of SEQ ID NO: 39.

80. An antibody or antigen binding fragment thereof that binds to an epitope of CA9-3 comprising a PTM, wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 71, and three light chain CDRs (LCDR1, LCDR2, and LCDR3) contained within a light chain variable region (LCVR) comprising the amino acid sequence of SEQ ID NO: 75.

81. The antibody or antigen binding fragment of claim 80, wherein the antibody or antigen binding fragment comprises HCDR1-HCDR2-HCDR3-LCDR1-LCDR2-LCDR3 domains comprising the amino sequences of SEQ ID NOs: 72-73-74-76-77-78, respectively.

82. The antibody or antigen binding fragment of claim 80 or 81, wherein the antibody or antigen-binding fragment thereof comprises a HCVR comprising the amino acid sequence of SEQ ID NO: 71, and a LCVR comprising the amino acid sequence of SEQ ID NO: 75.

83. The antibody or antigen binding fragment of any one of claims 80-82, wherein the epitope comprises the amino acid sequence of SEQ ID NO: 45.

84. An antibody or antigen binding fragment thereof that binds to an epitope of TFRC-5 comprising a PTM, wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 47, and three light chain CDRs (LCDR1, LCDR2, and LCDR3) contained within a light chain variable region (LCVR) comprising the amino acid sequence of SEQ ID NO: 51.

85. The antibody or antigen binding fragment of claim 84, wherein the antibody or antigen binding fragment comprises HCDR1-HCDR2-HCDR3-LCDR1-LCDR2-LCDR3 domains comprising the amino sequences of SEQ ID NOs: 48-49-50-52-53-54, respectively.

86. The antibody or antigen binding fragment of claim 84 or 85, wherein the antibody or antigen-binding fragment thereof comprises a HCVR comprising the amino acid sequence of SEQ ID NO: 47, and a LCVR comprising the amino acid sequence of SEQ ID NO: 51.

87. The antibody or antigen binding fragment of any one of claims 84-86, wherein the epitope comprises the amino acid sequence of SEQ ID NO: 40.

88. An antibody or antigen binding fragment thereof that binds to an epitope of TFRC-1 or TFRC-2 comprising a PTM, wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 55, and three light chain CDRs (LCDR1, LCDR2, and LCDR3) contained within a light chain variable region (LCVR) comprising the amino acid sequence of SEQ ID NO: 59.

89. The antibody or antigen binding fragment of claim 88, wherein the antibody or antigen binding fragment comprises HCDR1-HCDR2-HCDR3-LCDR1-LCDR2-LCDR3 domains comprising the amino sequences of SEQ ID NOs: 56-57-58-60-61-62, respectively.

90. The antibody or antigen binding fragment of claim 88 or 89, wherein the antibody or antigen-binding fragment thereof comprises a HCVR comprising the amino acid sequence of SEQ ID NO: 55, and a LCVR comprising the amino acid sequence of SEQ ID NO: 59.

91. The antibody or antigen binding fragment of any one of claims 88-90, wherein the epitope comprises the amino acid sequence of SEQ ID NO: 36 or SEQ ID NO: 37.

92. An antibody or antigen binding fragment thereof that binds to an epitope of SPINK1-1 comprising a PTM, wherein the antibody or antigen-binding fragment thereof comprises three heavy chain complementarity determining regions (CDRs) (HCDR1, HCDR2, and HCDR3) contained within a heavy chain variable region (HCVR) comprising the amino acid sequence of SEQ ID NO: 63, and three light chain CDRs (LCDR1, LCDR2, and LCDR3) contained within a light chain variable region (LCVR) comprising the amino acid sequence of SEQ ID NO: 67.

93. The antibody or antigen binding fragment of claim 92, wherein the antibody or antigen binding fragment comprises HCDR1-HCDR2-HCDR3-LCDR1-LCDR2-LCDR3 domains comprising the amino sequences of SEQ ID NOs: 64-65-66-68-69-70, respectively.

94. The antibody or antigen binding fragment of claim 92 or 93, wherein the antibody or antigen-binding fragment thereof comprises a HCVR comprising the amino acid sequence of SEQ ID NO: 63, and a LCVR comprising the aminoi acid sequence of SEQ ID NO: 67.

95. The antibody or antigen binding fragment of any one of claims 92-94, wherein the epitope comprises the amino acid sequence of SEQ ID NO: 29.

96. A pharmaceutical composition comprising an antibody or antigen-binding fragment thereof of any one of claims 76-95, and a pharmaceutically acceptable carrier or diluent.

97. A unit dosage form comprising a container containing an antibody or antigen-binding fragment thereof of any one of claims 76-95.

98. The unit dosage form of claim 97, wherein the container is a vial, a syringe, a prefilled syringe, or an autoinjector.

99. A kit comprising a container containing an antibody or antigen-binding fragment thereof of any one of claims 76-95, and instructions for use of the antibody or antigen-binding fragment for the treatment of small cell lung cancer.

100. A nucleic acid molecule encoding an antibody or antigen-binding fragment of any one of claims 76-95.

101. A recombinant expression vector comprising the nucleic acid molecule of claim 100.

102. A pair of nucleic acid molecules encoding heavy and light chain variable regions of an antibody or antigen-binding fragment of any one of claims 76-95, wherein the pair of nucleic acid molecules comprises a first nucleic acid molecule encoding a heavy chain variable region of the antibody or antigen-binding fragment, and a second nucleic acid molecule encoding a light chain variable region of the antibody or antigen-binding fragment.

103. A pair of recombinant expression vectors comprising the pair of nucleic acid molecules, respectively, of claim 102.

104. An isolated host cell comprising an antibody or antigen-binding fragment of any one of claims 76-95, a nucleic acid molecule of claim 100, a recombinant expression vector of claim 101, a pair of nucleic acid molecules of claim 102, or a pair of recombinant expression vectors of claim 103.

105. The isolated host cell of claim 104 that is a mammalian host cell.


SEQUENCE LISTING (86 sequences)

SEQ ID NO: 1 - protein, 6 aa, synthetic construct
  REGION 1..6: note = Synthetic
NVTDGS

SEQ ID NO: 2 - protein, 8 aa, synthetic construct
  REGION 1..8: note = Synthetic
GGCDGNSN

SEQ ID NO: 3 - protein, 7 aa, synthetic construct
  REGION 1..7: note = Synthetic
NAADSSV

SEQ ID NO: 4 - protein, 7 aa, synthetic construct
  REGION 1..7: note = Synthetic
GNKNSYR

SEQ ID NO: 5 - protein, 7 aa, synthetic construct
  REGION 1..7: note = Synthetic
RRQDSED

SEQ ID NO: 6 - protein, 7 aa, synthetic construct
  REGION 1..7: note = Synthetic
VERNSCN

SEQ ID NO: 7 - protein, 10 aa, synthetic construct
  REGION 1..10: note = Synthetic
YNELNGCTKI

SEQ ID NO: 8 - protein, 10 aa, synthetic construct
  REGION 1..10: note = Synthetic
LCFENRKRQT

SEQ ID NO: 9 - protein, 13 aa, synthetic construct
  REGION 1..13: note = Synthetic
TQKKSLNHRYQMG

SEQ ID NO: 10 - protein, 13 aa, synthetic construct
  REGION 1..13: note = Synthetic
VTEKNINGHQAKF

SEQ ID NO: 11 - protein, 10 aa, synthetic construct
  REGION 1..10: note = Synthetic
KAEGDGKMHI

SEQ ID NO: 12 - protein, 10 aa, synthetic construct
  REGION 1..10: note = Synthetic
IKRSDGSCAW

SEQ ID NO: 13 - protein, 10 aa, synthetic construct
  REGION 1..10: note = Synthetic
EKEVDSGNDI

SEQ ID NO: 14 - protein, 10 aa, synthetic construct
  REGION 1..10: note = Synthetic
DFPAARRLYW

SEQ ID NO: 15 - protein, 8 aa, synthetic construct
  REGION 1..8: note = Synthetic
NKVARAAA

SEQ ID NO: 16 - protein, 10 aa, synthetic construct
  REGION 1..10: note = Synthetic
LNDRVMRVEY

SEQ ID NO: 17 - protein, 13 aa, synthetic construct
  REGION 1..13: note = Synthetic
LYSARGDFFRATS

SEQ ID NO: 18 - protein, 6 aa, synthetic construct
  REGION 1..6: note = Synthetic
EEDSPR

SEQ ID NO: 19 - protein, 7 aa, synthetic construct
  REGION 1..7: note = Synthetic
LRNNGHS

SEQ ID NO: 20 - protein, 7 aa, synthetic construct
  REGION 1..7: note = Synthetic
GVDSSPR

SEQ ID NO: 21 - protein, 7 aa, synthetic construct
  REGION 1..7: note = Synthetic
LRDNHTH

SEQ ID NO: 22 - protein, 6 aa, synthetic construct
  REGION 1..6: note = Synthetic
  MOD_RES 4: note = iso-Aspartic acid
NVTXGS

SEQ ID NO: 23 - protein, 8 aa, synthetic construct
  REGION 1..8: note = Synthetic
  MOD_RES 4: note = iso-Aspartic acid
GGCXGNSN

SEQ ID NO: 24 - protein, 8 aa, synthetic construct
  REGION 1..8: note = Synthetic
  MOD_RES 6: note = iso-Aspartic acid
GGCDGXSN

SEQ ID NO: 25 - protein, 7 aa, synthetic construct
  REGION 1..7: note = Synthetic
  MOD_RES 4: note = iso-Aspartic acid
NAAXSSV

SEQ ID NO: 26 - protein, 7 aa, synthetic construct
  REGION 1..7: note = Synthetic
  MOD_RES 4: note = iso-Aspartic acid
GNKXSYR

SEQ ID NO: 27 - protein, 7 aa, synthetic construct
  REGION 1..7: note = Synthetic
  MOD_RES 4: note = iso-Aspartic acid
RRQXSED

SEQ ID NO: 28 - protein, 7 aa, synthetic construct
  REGION 1..7: note = Synthetic
  MOD_RES 4: note = iso-Aspartic acid
VERXSCN

SEQ ID NO: 29 - protein, 10 aa, synthetic construct
  REGION 1..10: note = Synthetic
  MOD_RES 5: note = iso-Aspartic acid
YNELXGCTKI

SEQ ID NO: 30 - protein, 10 aa, synthetic construct
  REGION 1..10: note = Synthetic
  MOD_RES 5: note = iso-Aspartic acid
LCFEXRKRQT

SEQ ID NO: 31 - protein, 13 aa, synthetic construct
  REGION 1..13: note = Synthetic
  MOD_RES 7: note = iso-Aspartic acid
TQKKSLXHRYQMG

SEQ ID NO: 32 - protein, 13 aa, synthetic construct
  REGION 1..13: note = Synthetic
  MOD_RES 7: note = iso-Aspartic acid
VTEKNIXGHQAKF

SEQ ID NO: 33 - protein, 10 aa, synthetic construct
  REGION 1..10: note = Synthetic
  MOD_RES 5: note = iso-Aspartic acid
KAEGXGKMHI

SEQ ID NO: 34 - protein, 10 aa, synthetic construct
  REGION 1..10: note = Synthetic
  MOD_RES 5: note = iso-Aspartic acid
IKRSXGSCAW

SEQ ID NO: 35 - protein, 10 aa, synthetic construct
  REGION 1..10: note = Synthetic
  MOD_RES 5: note = iso-Aspartic acid
EKEVXSGNDI

SEQ ID NO: 36 - protein, 10 aa, synthetic construct
  REGION 1..10: note = Synthetic
  MOD_RES 6: note = citrulline
DFPAAXRLYW

SEQ ID NO: 37 - protein, 10 aa, synthetic construct
  REGION 1..10: note = Synthetic
  MOD_RES 7: note = citrulline
DFPAARXLYW

SEQ ID NO: 38 - protein, 8 aa, synthetic construct
  REGION 1..8: note = Synthetic
  MOD_RES 5: note = citrulline
LYSAXGDF

SEQ ID NO: 39 - protein, 11 aa, synthetic construct
  REGION 1..11: note = Synthetic
  MOD_RES 7: note = citrulline
LNDRVMXVEYC

SEQ ID NO: 40 - protein, 9 aa, synthetic construct
  REGION 1..9: note = Synthetic
  MOD_RES 4: note = citrulline
LNDXVMRVC

SEQ ID NO: 41 - protein, 8 aa, synthetic construct
  REGION 1..8: note = Synthetic
  MOD_RES 5: note = citrulline
GDFFXATS

SEQ ID NO: 42 - protein, 8 aa, synthetic construct
  REGION 1..8: note = Synthetic
  MOD_RES 5: note = citrulline
NKVAXAAA

SEQ ID NO: 43 - protein, 6 aa, synthetic construct
  REGION 1..6: note = Synthetic
  MOD_RES 3: note = iso-Aspartic acid
EEXSPR

SEQ ID NO: 44 - protein, 7 aa, synthetic construct
  REGION 1..7: note = Synthetic
  MOD_RES 4: note = iso-Aspartic acid
LRNXGHS

SEQ ID NO: 45 - protein, 7 aa, synthetic construct
  REGION 1..7: note = Synthetic
  MOD_RES 3: note = iso-Aspartic acid
GVXSSPR

SEQ ID NO: 46 - protein, 7 aa, synthetic construct
  REGION 1..7: note = Synthetic
  MOD_RES 4: note = iso-Aspartic acid
LRDXHTH

SEQ ID NO: 47 - protein, 115 aa, Homo sapiens
QVQLVESGGGVVQPGRSLRLSCAASGFTFSTYGMHWVRQAPGKGLQWVAQISNDGSSKYYGDSVKGRFTISRDNSKNTLYLQMNSLRVEDTGVYYCASSGSSGTWGQGTQVTVSS

SEQ ID NO: 48 - protein, 8 aa, Homo sapiens
GFTFSTYG

SEQ ID NO: 49 - protein, 8 aa, Homo sapiens
ISNDGSSK

SEQ ID NO: 50 - protein, 8 aa, Homo sapiens
ASSGSSGT

SEQ ID NO: 51 - protein, 107 aa, Homo sapiens
EIVLTQSPATLFLFPGERVTFSCRASQSVNNYLAWYQQKPGQAPRLLIYDVSNRATGIPARFSGSGSGTDFTLTISSLDPEDFAVYFCQQRSNWPLTFGGGTKVEIK

SEQ ID NO: 52 - protein, 6 aa, Homo sapiens
QSVNNY

SEQ ID NO: 53 - skipped

SEQ ID NO: 54 - protein, 10 aa, Homo sapiens
QQRSNWPLTF

SEQ ID NO: 55 - protein, 118 aa, Homo sapiens
EVQLLESGGGLAQPGGSLRLSCGASGFTFSSHALSWVRLAPGKGLEWVSAISGSGDRTYYADSVKGRFTISRDNSKNMLYLQMNSLRGEDTAVYYCAKEATESYATSWGQGTLVTVSS

SEQ ID NO: 56 - protein, 8 aa, Homo sapiens
GFTFSSHA

SEQ ID NO: 57 - protein, 8 aa, Homo sapiens
ISGSGDRT

SEQ ID NO: 58 - protein, 11 aa, Homo sapiens
AKEATESYATS

SEQ ID NO: 59 - protein, 106 aa, Homo sapiens
DIQMTQSPSTLSASVGDRVTITCRASQSISSWLAWYQQKPGKAPNLLISKAFSVESGVPSTFSGSGSGTEFTLTISSLQPRDSASYYCQQYDSGWTFGQGTKVEIK

SEQ ID NO: 60 - protein, 6 aa, Homo sapiens
QSISSW

SEQ ID NO: 61 - skipped

SEQ ID NO: 62 - protein, 8 aa, Homo sapiens
QQYDSGWT

SEQ ID NO: 63 - protein, 125 aa, Homo sapiens
QAGLLESGGGVVQPGRSLRLSCAASGFTFSSYGMHWVRQAPGKGLEWVAVIWYDGSNKYYADSVKGRFTISRDNSKNTLYLQMNSLRAEDTAVYYCARGGGITGTVSRKQGMDVWGQGTLVTVSS

SEQ ID NO: 64 - protein, 8 aa, Homo sapiens
GFTFSSYG

SEQ ID NO: 65 - protein, 8 aa, Homo sapiens
IWYDGSNK

SEQ ID NO: 66 - protein, 18 aa, Homo sapiens
ARGGGITGTVSRKQGMDV

SEQ ID NO: 67 - protein, 107 aa, Homo sapiens
AIQLTQSPSSLSASVGDRVTITCRASQGISSYLAWYQQKPGKAPKLLMYGASTLQSGVPSRFSGSGSGTDFTLTISSLQPEDFATYYCQQVNSYPLTFGGGTKVEIK

SEQ ID NO: 68 - protein, 6 aa, Homo sapiens
QGISSY

SEQ ID NO: 69 - skipped

SEQ ID NO: 70 - protein, 9 aa, Homo sapiens
QQVNSYPLT

SEQ ID NO: 71 - protein, 123 aa, Homo sapiens
QVQLVESGGGLVQPGGSLRLSCAASGFTFSSYWMSWVRQAPGKGLEWVANIKQDGSEEYYVDSVKGRFTISRDNAKSLLYLQMNSLRPEDTAVYYCAGDSLPSSGWEGYFQHWGQGTLVTVSS

SEQ ID NO: 72 - protein, 8 aa, Homo sapiens
GFTFSSYW

SEQ ID NO: 73 - protein, 8 aa, Homo sapiens
IKQDGSEE

SEQ ID NO: 74 - protein, 16 aa, Homo sapiens
AGDSLPSSGWEGYFQH

SEQ ID NO: 75 - protein, 113 aa, Homo sapiens
DIVMTQSPVTLAVSLGERATINCKSSQSVLYSSNNKNYLAWYQQKPGQPPKLLIYWASTRESGVPDRFSGSGSGTDFTLTISSLQAEDVAVYYCQQYYSSPRTFGQGTKVEIK

SEQ ID NO: 76 - protein, 12 aa, Homo sapiens
QSVLYSSNNKNY

SEQ ID NO: 77 - skipped

SEQ ID NO: 78 - protein, 9 aa, Homo sapiens
QQYYSSPRT

SEQ ID NO: 79 - protein, 126 aa, Homo sapiens
QVQLVESGGGVVQPGRSLRLSCAASGFTFNSFALMWVRQAPGKGLEWVAVISNDGSNTYYADSVKGRFTISRDNSKNTLYLQMNSLRAEDTAVYYCARGSQYYGSGRYSRKQFDYWGQGTMVTVSS

SEQ ID NO: 80 - protein, 8 aa, Homo sapiens
GFTFNSFA

SEQ ID NO: 81 - protein, 8 aa, Homo sapiens
ISNDGSNT

SEQ ID NO: 82 - protein, 19 aa, Homo sapiens
ARGSQYYGSGRYSRKQFDY

SEQ ID NO: 83 - protein, 113 aa, Homo sapiens
DIQMTQSPLSLPVTPGEPASISCRSSQSLLHADDGNTYLDWYLQKPGQSPQLLIYTLSSRASGVPDRFSGSGSGTNFTLRISRVEAEDVGVYYCMQRIESPLTFGGGTKVEIK

SEQ ID NO: 84 - protein, 12 aa, Homo sapiens
QSLLHADDGNTY

SEQ ID NO: 85 - skipped

SEQ ID NO: 86 - protein, 9 aa, Homo sapiens
MQRIESPLT